(19) United States
(12) Patent Application Publication
(10) Pub. No.: US 2009/0286301 A1
(43) Pub. Date: Nov. 19, 2009

(54) EXTRACTION OF BIOMOLECULAR COMPLEXES ASSISTED BY ALTERNATING HYDROSTATIC PRESSURE

(75) Inventors: Feng Tao, Germantown, MD (US); Richard Schumacher, Taunton, MA (US); Nathan Lawrence, South Easton, MA (US); Vera Gross, Newtonville, MA (US); Gary Smejkal, Hudson, NH (US); Alexander Lazarev, Lexington, MA (US); Rui Han, Boyds, MD (US)

Correspondence Address: LANDO & ANASTASI, LLP, ONE MAIN STREET, SUITE 1100, CAMBRIDGE, MA, 02142, US

(73) Assignee: PRESSURE BIOSCIENCES INC., South Easton, MA (US)

(21) Appl. No.: 12/437,162

(22) Filed: May 7, 2009

Related U.S. Application Data
(60) Provisional application No. 61/051,133, filed on May 7, 2008.

(51) Int. Cl. C12N 9/20; C12N 9/24; C12N 9/50; C12N 5/06

(52) U.S. Cl. 435/198; 435/200; 435/219; 435/375; 435/317.1

(57) ABSTRACT

Extraction methods that allow a molecular complex (e.g., an organelle) to be extracted from a sample by employing pressure cycling are described.

### CROSS REFERENCE TO RELATED APPLICATIONS

This application claims priority to U.S. Application Ser. No. 61/051,133, filed on May 7, 2008. The disclosure of the prior application is considered part of (and is incorporated by reference in) the disclosure of this application.

### FEDERALLY SPONSORED RESEARCH OR DEVELOPMENT

The U.S. Government may have certain rights in this invention pursuant to Grant No. GM079059 awarded by NIGMS, National institute of Health.

### BACKGROUND

Biological samples are generally highly heterogeneous, diverse, and complex. To study a biological sample at the molecular level, a sample preparation process may need to be performed. This process not only permits the release of the target analyte(s) into solution, but, in some instances, also involves dissolution of the analyte. In many cases, structural integrity and biological activity of the target analyte need to be maintained, e.g., for subsequent sample manipulations or analysis.

### SUMMARY

The present disclosure provides, inter alia, extraction methods that allow a molecular complex (e.g., an organelle) to be extracted from a sample by employing pressure cycling. Cycles of pressure (e.g., hydrostatic pressure), for example, from ambient to high pressure and back down to ambient (pressure cycling), can disrupt cells and tissues more efficiently than non-cycling application of pressure (as described herein; see also U.S. Pat. Nos. 6,274,726; 6,120,985; 6,270,723; and 6,696,019).

The present disclosure describes novel methods of using alternating hydrostatic pressure in the extraction of an entity (e.g., molecular complex) from a biological sample. This extraction reaction can be carried out in closed devices, which not only hold the sample and extraction buffer(s), but which may also be equipped with special spatial features and/or physical structures suitable for hydrostatic pressure cycles. The alternating hydrostatic pressure applied to the sample may be defined based on the structural features of the sample as well as the structural features of the targets (e.g., a molecular complex, e.g., an organelle). In some aspects, pressure can be used to disrupt cell membrane and connective tissue structures, but to leave a molecular complex of interest intact. In some aspects, pressure can be used to disrupt some molecular complexes (e.g., a pressure-sensitive molecular complex), but leave intact other molecular complexes (e.g., a pressure-tolerant molecular complex), e.g., a molecular complex of interest (e.g., an organelle). For example, mitochondria can be maintained intact, while membrane protein complexes on/in the outer cellular membrane are disrupted.

The molecular complexes to be extracted can be, e.g., intact sub-cellular organelles, fragments of organelles, fragments of biological membrane, membrane structures other than organelles (e.g. microsomes), protein complexes (e.g., such as channel proteins, protein-nucleic acid, protein and protein cofactor, protein-small molecule or protein-lipid complexes), viruses, or a subset of different cells in a sample. The characteristics of molecular complexes include the involvement of two or more molecules and/or two types (or more) of molecules in each complex. The molecular complexes that can be extracted by the methods described herein include any biomolecules, such as protein-protein, protein-lipid, protein-nucleic acid, protein-peptide, protein-small molecules, nucleic acid-small molecules, and lipid-lipid complexes.

The methods described herein can produce, e.g., extracted fractions of molecular complexes. For example, a fraction of a certain type of molecular complex produced by the methods described herein may contain only or predominantly one type of a molecular complex (e.g., the fraction is enriched for one type of molecular complex, e.g., the molecular complex makes up about 10%, about 20%, about 30%, about 40%, about 50%, about 60%, about 70%, about 80%, about 90%, about 92%, about 93%, about 94%, about 95%, about 96%, about 97%, about 98%, about 99%, about 100% of the fraction). For example, purified mitochondria with little or no contamination from other organelles, multiplicity of bacterial cells of a certain strain, and a multiplicity of viral particles of a particular species can be prepared. In other embodiments, molecular complex fractions that contain several types of molecular complexes, such as a subcellular fraction with a particular buoyant density, e.g., which may contain fragments of plasma membrane and nuclear membrane in addition to intact mitochondria, etc., can be prepared. Such fractions are typically produced by conventional subcellular fractionation techniques. Several embodiments of the present invention describe a combination of several orthogonal methods of cell disruption and extraction resulting in selective disassembly of several undesirable types of molecular complexes contained in a particular heterogeneous fraction, which leads to enrichment for the complex(es) of interest. In some embodiments, the complexes retain their original composition, or at least a part thereof. For example, fragments of organelles, membrane fragments, and protein-lipid complexes (e.g., multimeric protein complexes associated with lipid bilayer, such as transporters or transmembrane channels, such as VDAC/Porin, etc.) can be extracted with the methods described herein.

Pressure can be used with specifically-designed extraction buffers, or with currently available buffers that are suitable for pressure cycling. Some of the buffers may contain lytic enzymes, surfactants, and/or other kinds of chemicals, such as polymers having multi-functional groups, e.g., zwitterionic detergents, e.g. 3-[(3-Cholamidopropyl)dimethylammonio]-1-propanesulfonate (CHAPS), non-detergent sulfobetaines (NDSB), choline phosphatides, n-Octyl-b-D-Glucopyranoside and lauryldimethylamine oxide. Specific combinations of pressure and temperature can be used to control a particular thermodynamic state of matter, e.g., application of pressure P₁ at temperature T₁ followed by application of pressure P₂ at temperature T₂, and so on, may be employed. Since biological samples often contain a mixture of different types of entities, e.g., cells, microbes, and microstructures, the extraction methods described herein may target specific group(s) or types of complexes depending on the particular pressure and/or other condition sensitivities of the complexes.

In one aspect, the disclosure features a method of extracting a molecular complex from a sample. The method includes providing a mixture at a first pressure, P₀, wherein the mixture comprises a sample and a liquid phase, and wherein the sample contains the molecular complex;

exposing the mixture to a second pressure, P₁, wherein P₁ pressure is greater than P₀;

exposing the mixture to a third pressure, P₂, wherein P₂ is less than P₁; and

fractionating the mixture, thereby extracting the molecular complex from the sample.

In some embodiments, the fractionating includes centrifugation (e.g., buoyant density accumulation (BDA), or sucrose gradient separation), chromatography (e.g., HPLC, affinity binding chromatography, or SEC), electrophoresis, filtration, or dialysis.

In some embodiments, the molecular complex remains intact (e.g., after performing the steps of the method).

In some embodiments, the structural integrity of the molecular complex is maintained (e.g., after performing the steps of the method). For example, the molecular complex appears morphologically normal (e.g., upon visual inspection, the molecular complex appears substantially the same as it did prior to the extracting). As another example, if the molecular complex is bound by a membrane, the membrane remains substantially intact, e.g., if the molecular complex is bound by both inner and outer membranes, the inner and/or outer membrane remain intact. If the molecular complex includes a protein complex of two or more proteins (e.g., the molecular complex is a membrane channel, membrane pore, signal transduction complex), at least two (or all) of the proteins remain associated with each other. In some embodiments, another component of the complex (e.g., a non-protein co-factor, lipid, nucleic acid, small molecule) remains associated with a protein component of the complex. Additional examples are provided herein.

In some embodiments, a biological activity of the molecular complex is maintained (e.g., after performing the steps of the method). For example, if the molecular complex is a mitochondrion, the mitochondrion maintains mitochondrial respiration, e.g., mitochondrial respiration can be detected, e.g., as measured by a respiratory control ratio and/or an ADP/O ratio. As another example, if the molecular complex is a peroxisome, it maintains the ability to metabolize fatty acids or break down peroxide. If the molecular complex is a lysosome, the lysosome maintains its inner acidic pH and/or ability to pump protons across its membrane.

In some embodiments, the molecular complex is pressure-tolerant.

In some embodiments, P₁ is between about 1,000 psi and about 100,000 psi.

In some embodiments, the rate of change from P₁ to P₂ is between about 1 and about 1,000 psi/millisecond.

In some embodiments, P₀ is between about 14.7 psi to about 15,000 psi.

In some embodiments, P₁ is between about 1,000 psi and about 60,000 psi.

In some embodiments, P₂ is about equal to P₀.

In some embodiments, P₂ is greater than P₀.

In some embodiments, P₂ is less than P₀.

In some embodiments, the pressure is changed from P₂ to a fourth pressure, P₃.

In some embodiments, P₃ is greater than P₂.

In some embodiments, P₃ is less than P₂.

In some embodiments, P₃ is about equal to P₁.

In some embodiments, P₃ greater than P₁.

In some embodiments, P₃ is less than P₁.

In some embodiments, the sample is exposed to a pressure cycle, wherein P₀, P₁, and P₂ comprise the pressure cycle.

In some embodiments, the number of pressure cycles ranges between about 1 cycle to about 250 cycles.

In some preferred embodiments, the pressure cycle includes:

providing a mixture at about 101.3 KPa, subjecting the mixture to an elevated pressure of about 50 MPa held for 5 seconds, and subjecting the mixture to about 101.3 KPa for 10 seconds.

In some embodiments, the pressure cycle is repeated 5 times.

In other preferred embodiments, the pressure cycle includes:

providing a mixture at about 100 MPa, subjecting the mixture to an elevated pressure of about 250 MPa held for 10 seconds, subjecting the mixture to a pressure of about 200 MPa held for 5 seconds and subjecting the mixture to about 100 MPa held for 5 seconds.

In some embodiments, the pressure cycle is repeated 10 times.

In some embodiments, the pressure is applied as hydraulic or pneumatic pressure.

In some embodiments, the method is performed at a temperature between about 0° C. and about +100° C.

In some embodiments, the liquid phase comprises a buffer.

In some embodiments, the buffer comprises phosphate buffered saline (PBS).

In some embodiments, the buffer comprises HEPES buffer.

In some embodiments, the buffer comprises a mitochondrial isolation buffer (MIB).

In some embodiments, the liquid phase comprises a solvent.

In some embodiments, the liquid phase comprises a protease inhibitor, a DNAse inhibitor, or an RNAse inhibitor.

In some embodiments, the liquid phase comprises a protease, a DNAse, an RNAse, or a lipase.

In some embodiments, the sample is of biological or of synthetic (e.g., man-made) origin.

In some embodiments, the sample is of biological origin and is from a mammalian (e.g., human or domesticated animal), fungal, bacterial, viral, or plant source.

In some embodiments, the sample includes a cell, a membrane (e.g., a lipid membrane, e.g., a lipid bilayer), a biological sample (e.g., tissue sample, e.g., adipose tissue, liver, kidney, skin, pancreas, stomach, intestine, colon, breast, ovary, uterine, prostate, bone, tendon, cartilage, hair, nail, tooth, heart, brain, lung, skin, nerves, biopsy, etc., blood, urine, milk, semen, saliva, mucus, other bodily fluids and solids), or a collection of cells (e.g., blood, semen, mucus, saliva, tissue biopsy).

In some embodiments, the sample size is from about 10 microliters to about 50 milliliters.

In some embodiments, the sample includes a liquid.

In some embodiments, the liquid includes a body fluid (e.g., blood, serum, urine, or spinal fluid).

In some embodiments, the sample includes a soft tissue (e.g., liver, kidney, ovary, pancreas, or brain).

In some embodiments, the sample includes a hard tissue (e.g., muscle, intestine, heart, adipose, skin, hair, finger nail, bone, or cartilage).

In some embodiments, the molecular complex includes an organelle, a protein complex (e.g., that contains two or more proteins), a membrane channel, a membrane pore, a transcription factor complex, a signal transduction complex, or a sub-organelle structure (e.g., an inner membrane and its contents of a mitochondrion).

In some embodiments, the molecular complex includes an organelle and the organelle is a mitochondrion, nucleus, Golgi apparatus, chloroplast, endoplasmic reticulum (ER), vacuole, acrosome, centriole, cilium, glyoxysome, hydrogenosome, lysosome, melanosome, mitosome, myofibril, nucleolus, parenthesome, peroxisome, ribosome, proteosome, microsome, or vesicle.

In some embodiments, a fragment (e.g., a fragment of an ER or a Golgi complex, or a mitochondrion stripped of its outer membrane) of the organelle is extracted.

In some embodiments, the molecular complex includes a protein-protein, a protein-lipid, a protein-nucleic acid, a protein-peptide, a protein-small molecule, a nucleic acid-small molecule, or a lipid-protein complex.

In some embodiments, the extracted molecular complex is used for genomic analysis.

In some embodiments, the extracted molecular complex is used for proteomic analysis.

In some embodiments, the extracted molecular complex is used for diagnostics (e.g., of a medical disease or condition).

In some embodiments, the extracted molecular complex is further analyzed.

In some preferred embodiments, the extracted molecular complex is analyzed by two-dimensional gel electrophoresis, one-dimensional gel electrophoresis, Western blotting, ELISA, protein or peptide mass fingerprinting (e.g., using MALDI-TOF/TOF), multi-dimensional electrophoresis (e.g., solution phase isoelectric focusing followed by two-dimensional gel electrophoresis of concentrated pI fractions), mass spectrometry (MALDI-MS, LC-MS/MS, MALDI-TOF MS, or LC-ESI-MS/MS), PCR, RT-PCR, microarrays, thin-layer chromatography, liquid chromatography, gas chromatography, GC/MS, electron microscopy, fluorescent microscopy, or surface analysis methods.

In some embodiments, the extracted molecular complex is analyzed for the presence of a component (e.g., a protein, an enzyme, a DNA sequence (e.g., a mutation, methylation, and other adduct), an RNA sequence (e.g., a mutation, maturation), a metabolite).

In some embodiments, the method further includes a purification step.

In some embodiments, the purification step includes a centrifugation step or a filtration step.

In some embodiments, the centrifugation step or the filtration step is performed before the extraction.

In some embodiments, the centrifugation step or the filtration step is performed after the extraction.

In some embodiments, the method includes an additional fractionation step.

In some embodiments, the fractionation step is performed is performed before the extraction.

In some embodiments, the fractionation step is performed after the extraction.

In some embodiments, the fractionation step includes centrifugation, chromatography including buoyant density accumulation (BDA), sucrose gradient separation, HPLC, affinity binding chromatography, SEC, electrophoresis, filtration, or dialysis.

In one aspect, the methods described herein can be used to deplete (e.g., selectively deplete) a component from a sample (e.g., reduce the amount of the component in the sample by about 10%, about 20%, about 30%, about 40%, about 50%, about 60%, about 70%, about 80%, about 90%, about 95%, or about 99%). The method includes

providing a mixture at a first pressure, P₀, wherein the mixture comprises a sample and a liquid phase, and wherein the sample contains the component;

exposing the mixture to a second pressure, P₁, wherein P₁ pressure is greater than P₀; exposing the mixture to a third pressure, P₂, wherein P₂ is less than P₁; and

fractionating the mixture, thereby depleting the component from the sample.

In some embodiments, the sample is or includes a tissue sample (e.g., bone or muscle, e.g., skeletal or cardiac muscle).

In some embodiments, the component is a protein, e.g., a blood-derived protein.

In some embodiments, the component is a contaminant, e.g., another cell type. For example, the component is a bacterial cell in a sample that includes eukaryotic cells.

In some embodiments, the structural integrity of the sample is maintained (e.g., other than the depletion of the component) (e.g., after performing the steps of the method). For example, the sample appears morphologically normal (e.g., upon visual inspection, the sample appears substantially the same as it did prior to the depleting).

In some embodiments, a biological activity of the sample is maintained (e.g., after performing the steps of the method).

In some embodiments, the component is pressure-sensitive.

In some embodiments, the fractionating includes centrifugation (e.g., buoyant density accumulation (BDA), or sucrose gradient separation), chromatography (e.g., HPLC, affinity binding chromatography, or SEC), electrophoresis, filtration, or dialysis.

In some embodiments, P₁ is between about 1,000 psi and about 100,000 psi.

In some embodiments, the rate of change from P₁ to P₂ is between about 1 and about 1,000 psi/millisecond.

In some embodiments, P₀ is between about 14.7 psi to about 15,000 psi.

In some embodiments, P₁ is between about 1,000 psi and about 60,000 psi.

In some embodiments, P₂ is about equal to P₀.

In some embodiments, P₂ is greater than P₀.

In some embodiments, P₂ is less than P₀.

In some embodiments, the pressure is changed from P₂ to a fourth pressure, P₃.

In some embodiments, P₃ is greater than P₂.

In some embodiments, P₃ is less than P₂.

In some embodiments, P₃ is about equal to P₁.

In some embodiments, P₃ greater than P₁.

In some embodiments, P₃ is less than P₁.

In some embodiments, the sample is exposed to a pressure cycle, wherein P₀, P₁, and P₂ comprise the pressure cycle.

In some embodiments, the number of pressure cycles ranges between about 1 cycle to about 250 cycles.

In some preferred embodiments, the pressure cycle includes:

providing a mixture at about 101.3 KPa, subjecting the mixture to an elevated pressure of about 50 MPa held for 5 seconds, and subjecting the mixture to about 101.3 KPa for 10 seconds.

In some embodiments, the pressure cycle is repeated 5 times.

In other preferred embodiments, the pressure cycle includes:

providing a mixture at about 100 MPa, subjecting the mixture to an elevated pressure of about 250 MPa held for 10 seconds, subjecting the mixture to a pressure of about 200 MPa held for 5 seconds and subjecting the mixture to about 100 MPa held for 5 seconds.

In some embodiments, the pressure cycle is repeated 10 times.

In some embodiments, the pressure is applied as hydraulic or pneumatic pressure.

In some embodiments, the method is performed at a temperature between about 0° C. and about +100° C.

In some embodiments, the liquid phase comprises a buffer.

In some embodiments, the buffer comprises phosphate buffered saline (PBS).

In some embodiments, the buffer comprises HEPES buffer.

In some embodiments, the buffer comprises a mitochondrial isolation buffer (MIB).

In some embodiments, the liquid phase comprises a solvent.

In some embodiments, the liquid phase comprises a protease inhibitor, a DNAse inhibitor, or an RNAse inhibitor.

In some embodiments, the liquid phase comprises a protease, a DNAse, an RNAse, or a lipase.

In some embodiments, the sample is of biological or of synthetic (e.g., man-made) origin.

In some embodiments, the sample is of biological origin and is from a mammalian (e.g., human or domesticated animal), fungal, bacterial, viral, or plant source.

In some embodiments, the sample includes a cell, a membrane (e.g., a lipid membrane, e.g., a lipid bilayer), a biological sample (e.g., tissue sample, e.g., adipose tissue, liver, kidney, skin, pancreas, stomach, intestine, colon, breast, ovary, uterine, prostate, bone, tendon, cartilage, hair, nail, tooth, heart, brain, lung, skin, nerves, biopsy, etc., blood, urine, milk, semen, saliva, mucus, other bodily fluids and solids), or a collection of cells (e.g., blood, semen, mucus, saliva, tissue biopsy).

In some embodiments, the sample size is from about 10 microliters to about 50 milliliters.

In some embodiments, the sample includes a liquid.

In some embodiments, the liquid includes a body fluid (e.g., blood, serum, urine, or spinal fluid).

In some embodiments, the sample includes a soft tissue (e.g., liver, kidney, ovary, pancreas, or brain).

In some embodiments, the sample includes a hard tissue (e.g., muscle, intestine, heart, adipose, skin, hair, finger nail, bone, or cartilage).

In some embodiments, the sample is used for genomic analysis.

In some embodiments, the sample is used for proteomic analysis.

In some embodiments, the sample is used for diagnostics (e.g., of a medical disease or condition).

In some embodiments, the sample is further analyzed.

In some preferred embodiments, the sample is analyzed by two-dimensional gel electrophoresis, one-dimensional gel electrophoresis, Western blotting, ELISA, protein or peptide mass fingerprinting (e.g., using MALDI-TOF/TOF), multi-dimensional electrophoresis (e.g., solution phase isoelectric focusing followed by two-dimensional gel electrophoresis of concentrated pI fractions), mass spectrometry (MALDI-MS, LC-MS/MS, MALDI-TOF MS, or LC-ESI-MS/MS), PCR, RT-PCR, microarrays, thin-layer chromatography, liquid chromatography, gas chromatography, GC/MS, electron microscopy, fluorescent microscopy, or surface analysis methods.

In some embodiments, the component is analyzed for the presence of a second component (e.g., a protein, an enzyme, a DNA sequence (e.g., a mutation, methylation, and other adduct), an RNA sequence (e.g., a mutation, maturation), a metabolite).

In some embodiments, the method further includes a purification step.

In some embodiments, the purification step includes a centrifugation step or a filtration step.

In some embodiments, the centrifugation step or the filtration step is performed before the extraction.

In some embodiments, the centrifugation step or the filtration step is performed after the extraction.

In some embodiments, the method includes an additional fractionation step.

In some embodiments, the fractionation step is performed is performed before the extraction.

In some embodiments, the fractionation step is performed after the extraction.

In some embodiments, the fractionation step includes centrifugation, chromatography including buoyant density accumulation (BDA), sucrose gradient separation, HPLC, affinity binding chromatography, SEC, electrophoresis, filtration, or dialysis.

In one aspect, the methods described herein can be used to inactivate (e.g., selectively inactivate) a component in a sample (e.g., reduce the activity of the component in the sample by about 10%, about 20%, about 30%, about 40%, about 50%, about 60%, about 70%, about 80%, about 90%, about 95%, or about 99%). The method includes

providing a mixture at a first pressure, P₀, wherein the mixture comprises a sample and a liquid phase, and wherein the sample contains the component;

exposing the mixture to a second pressure, P₁, wherein P₁ pressure is greater than P₀;

exposing the mixture to a third pressure, P₂, wherein P₂ is less than P₁; thereby inactivating the component in the sample.

In some embodiments, the sample includes a plurality of cells (e.g., cells at different stages of differentiation, growth, or senescence, or a mixed population of cells of more than one cell type).

In some embodiments, the component is a contaminant, e.g., another cell type.

In some embodiments, the method synchronizes cells, e.g., by inactivating cells at a particular stage of differentiation, growth, or senescence, while leaving cells at a different stage of differentiation, growth, or senescence intact.

In some embodiments, the sample contains a mixed population of cells of more than one cell type, e.g., more than one type of prokaryotic cells (e.g., a population containing more than one bacterial cell type), more than one type of eukaryotic cells (e.g., a mixed population of testicular cells, e.g., that includes sperm cells), a population containing prokaryotic and eukaryotic cells. For example, one of the types of cells is inactivated.

In some embodiments, the structural integrity of the sample is maintained (e.g., other than the inactivation of the component) (e.g., after performing the steps of the method). For example, the sample appears morphologically normal (e.g., upon visual inspection, the sample appears substantially the same as it did prior to the inactivating).

In some embodiments, a biological activity of the sample is maintained (e.g., after performing the steps of the method).

In some embodiments, the component is pressure-sensitive.

In some embodiments, the fractionating includes centrifugation (e.g., buoyant density accumulation (BDA), or sucrose gradient separation), chromatography (e.g., HPLC, affinity binding chromatography, or SEC), electrophoresis, filtration, or dialysis.

In some embodiments, P₁ is between about 1,000 psi and about 100,000 psi.

In some embodiments, the rate of change from P₁ to P₂ is between about 1 and about 1,000 psi/millisecond.

In some embodiments, P₀ is between about 14.7 psi to about 15,000 psi.

In some embodiments, P₁ is between about 1,000 psi and about 60,000 psi.

In some embodiments, P₂ is about equal to P₀.

In some embodiments, P₂ is greater than P₀.

In some embodiments, P₂ is less than P₀.

In some embodiments, the pressure is changed from P₂ to a fourth pressure, P₃.

In some embodiments, P₃ is greater than P₂.

In some embodiments, P₃ is less than P₂.

In some embodiments, P₃ is about equal to P₁.

In some embodiments, P₃ greater than P₁.

In some embodiments, P₃ is less than P₁.

In some embodiments, the sample is exposed to a pressure cycle, wherein

P₀, P₁, and P₂ comprise the pressure cycle.

In some embodiments, the number of pressure cycles ranges between about 1 cycle to about 250 cycles.

In some preferred embodiments, the pressure cycle includes:

providing a mixture at about 101.3 KPa, subjecting the mixture to an elevated pressure of about 50 MPa held for 5 seconds, and subjecting the mixture to about 101.3 KPa for 10 seconds.

In some embodiments, the pressure cycle is repeated 5 times.

In other preferred embodiments, the pressure cycle includes:

providing a mixture at about 100 MPa, subjecting the mixture to an elevated pressure of about 250 MPa held for 10 seconds, subjecting the mixture to a pressure of about 200 MPa held for 5 seconds and subjecting the mixture to about 100 MPa held for 5 seconds.

In some embodiments, the pressure cycle is repeated 10 times.

In some embodiments, the pressure is applied as hydraulic or pneumatic pressure.

In some embodiments, the method is performed at a temperature between about 0° C. and about +100° C.

In some embodiments, the liquid phase comprises a buffer.

In some embodiments, the buffer comprises phosphate buffered saline (PBS).

In some embodiments, the buffer comprises HEPES buffer.

In some embodiments, the buffer comprises a mitochondrial isolation buffer (MIB).

In some embodiments, the liquid phase comprises a solvent.

In some embodiments, the liquid phase comprises a protease inhibitor, a DNAse inhibitor, or an RNAse inhibitor.

In some embodiments, the liquid phase comprises a protease, a DNAse, an RNAse, or a lipase.

In some embodiments, the sample is of biological or of synthetic (e.g., man-made) origin.

In some embodiments, the sample is of biological origin and is from a mammalian (e.g., human or domesticated animal), fungal, bacterial, viral, or plant source.

In some embodiments, the sample includes a cell, a membrane (e.g., a lipid membrane, e.g., a lipid bilayer), a biological sample (e.g., tissue sample, e.g., adipose tissue, liver, kidney, skin, pancreas, stomach, intestine, colon, breast, ovary, uterine, prostate, bone, tendon, cartilage, hair, nail, tooth, heart, brain, lung, skin, nerves, biopsy, etc., blood, urine, milk, semen, saliva, mucus, other bodily fluids and solids), or a collection of cells (e.g., blood, semen, mucus, saliva, tissue biopsy).

In some embodiments, the sample size is from about 10 microliters to about 50 milliliters.

In some embodiments, the sample includes a liquid.

In some embodiments, the liquid includes a body fluid (e.g., blood, serum, urine, or spinal fluid).

In some embodiments, the sample includes a soft tissue (e.g., liver, kidney, ovary, pancreas, or brain).

In some embodiments, the sample includes a hard tissue (e.g., muscle, intestine, heart, adipose, skin, hair, finger nail, bone, or cartilage).

In some embodiments, the sample is used for genomic analysis.

In some embodiments, the sample is used for proteomic analysis.

In some embodiments, the sample is used for diagnostics (e.g., of a medical disease or condition).

In some embodiments, the sample is further analyzed.

In some preferred embodiments, the sample is analyzed by two-dimensional gel electrophoresis, one-dimensional gel electrophoresis, Western blotting, ELISA, protein or peptide mass fingerprinting (e.g., using MALDI-TOF/TOF), multi-dimensional electrophoresis (e.g., solution phase isoelectric focusing followed by two-dimensional gel electrophoresis of concentrated pI fractions), mass spectrometry (MALDI-MS, LC-MS/MS, MALDI-TOF MS, or LC-ESI-MS/MS), PCR, RT-PCR, microarrays, thin-layer chromatography, liquid chromatography, gas chromatography, GC/MS, electron microscopy, fluorescent microscopy, or surface analysis methods.

In some embodiments, the sample is analyzed for the presence of a second component (e.g., a protein, an enzyme, a DNA sequence (e.g., a mutation, methylation, and other adduct), an RNA sequence (e.g., a mutation, maturation), a metabolite).

In some embodiments, the method further includes a purification step.

In some embodiments, the purification step includes a centrifugation step or a filtration step.

In some embodiments, the centrifugation step or the filtration step is performed before the extraction.

In some embodiments, the centrifugation step or the filtration step is performed after the extraction.

In some embodiments, the method includes an additional fractionation step.

In some embodiments, the fractionation step is performed is performed before the extraction.

In some embodiments, the fractionation step is performed after the extraction.

In some embodiments, the fractionation step includes centrifugation, chromatography including buoyant density accumulation (BDA), sucrose gradient separation, HPLC, affinity binding chromatography, SEC, electrophoresis, filtration, or dialysis.

In one aspect, the methods described herein can be used to extract a component from a sample. The method includes providing a mixture at a first pressure, P₀, wherein the mixture comprises a sample and a liquid phase, and wherein the sample contains the component; exposing the mixture to a second pressure, P₁, wherein P₁ pressure is greater than P₀;

exposing the mixture to a third pressure, P₂, wherein P₂ is less than P₁; and

fractionating the mixture, thereby extracting the component from the sample.

In some embodiments, the sample is or includes a tissue sample (e.g., bone or muscle, e.g., skeletal or cardiac muscle).

In some embodiments, the component is a protein, e.g., a blood-derived protein.

In some embodiments, the component is a nucleic acid, e.g., DNA or RNA (e.g., mRNA).

In some embodiments, the structural integrity of the component is maintained (e.g., after performing the steps of the method). For example, the component appears morphologically normal (e.g., upon visual inspection, the sample appears substantially the same as it did prior to the extracting). As another example, if the component is a protein, the protein is not denatured.

In some embodiments, a biological activity of the component is maintained (e.g., after performing the steps of the method). For example, if the component is a protein, the protein is able to interact with a binding partner or co-factor.

In some embodiments, the component is pressure-tolerant.

In some embodiments, the fractionating includes centrifugation (e.g., buoyant density accumulation (BDA), or sucrose gradient separation), chromatography (e.g., HPLC, affinity binding chromatography, or SEC), electrophoresis, filtration, or dialysis.

In some embodiments, P₁ is between about 1,000 psi and about 100,000 psi.

In some embodiments, the rate of change from P₁ to P₂ is between about 1 and about 1,000 psi/millisecond.

In some embodiments, P₀ is between about 14.7 psi to about 15,000 psi.

In some embodiments, P₁ is between about 1,000 psi and about 60,000 psi.

In some embodiments, P₂ is about equal to P₀.

In some embodiments, P₂ is greater than P₀.

In some embodiments, P₂ is less than P₀.

In some embodiments, the pressure is changed from P₂ to a fourth pressure, P₃.

In some embodiments, P₃ is greater than P₂.

In some embodiments, P₃ is less than P₂.

In some embodiments, P₃ is about equal to P₁.

In some embodiments, P₃ greater than P₁.

In some embodiments, P₃ is less than P₁.

In some embodiments, the sample is exposed to a pressure cycle, wherein P₀, P₁, and P₂ comprise the pressure cycle.

In some embodiments, the number of pressure cycles ranges between about 1 cycle to about 250 cycles.

In some preferred embodiments, the pressure cycle includes:

providing a mixture at about 101.3 KPa, subjecting the mixture to an elevated pressure of about 50 MPa held for 5 seconds, and subjecting the mixture to about 101.3 KPa for 10 seconds.

In some embodiments, the pressure cycle is repeated 5 times.

In other preferred embodiments, the pressure cycle includes:

providing a mixture at about 100 MPa, subjecting the mixture to an elevated pressure of about 250 MPa held for 10 seconds, subjecting the mixture to a pressure of about 200 MPa held for 5 seconds and subjecting the mixture to about 100 MPa held for 5 seconds.

In some embodiments, the pressure cycle is repeated 10 times.

In some embodiments, the pressure is applied as hydraulic or pneumatic pressure.

In some embodiments, the method is performed at a temperature between about 0° C. and about +100° C.

In some embodiments, the liquid phase comprises a buffer.

In some embodiments, the buffer comprises phosphate buffered saline (PBS).

In some embodiments, the buffer comprises HEPES buffer.

In some embodiments, the buffer comprises a mitochondrial isolation buffer (MIB).

In some embodiments, the liquid phase comprises a solvent.

In some embodiments, the liquid phase comprises a protease inhibitor, a DNAse inhibitor, or an RNAse inhibitor.

In some embodiments, the liquid phase comprises a protease, a DNAse, an RNAse, or a lipase.

In some embodiments, the sample is of biological or of synthetic (e.g., man-made) origin.

In some embodiments, the sample is of biological origin and is from a mammalian (e.g., human or domesticated animal), fungal, bacterial, viral, or plant source.

In some embodiments, the sample includes a cell, a membrane (e.g., a lipid membrane, e.g., a lipid bilayer), a biological sample (e.g., tissue sample, e.g., adipose tissue, liver, kidney, skin, pancreas, stomach, intestine, colon, breast, ovary, uterine, prostate, bone, tendon, cartilage, hair, nail, tooth, heart, brain, lung, skin, nerves, biopsy, etc., blood, urine, milk, semen, saliva, mucus, other bodily fluids and solids), or a collection of cells (e.g., blood, semen, mucus, saliva, tissue biopsy).

In some embodiments, the sample size is from about 10 microliters to about 50 milliliters.

In some embodiments, the sample includes a liquid.

In some embodiments, the liquid includes a body fluid (e.g., blood, serum, urine, or spinal fluid).

In some embodiments, the sample includes a soft tissue (e.g., liver, kidney, ovary, pancreas, or brain).

In some embodiments, the sample includes a hard tissue (e.g., muscle, intestine, heart, adipose, skin, hair, finger nail, bone, or cartilage).

In some embodiments, the component is used for genomic analysis.

In some embodiments, the component is used for proteomic analysis.

In some embodiments, the component is used for diagnostics (e.g., of a medical disease or condition).

In some embodiments, the component is further analyzed.

In some preferred embodiments, the component is analyzed by two-dimensional gel electrophoresis, one-dimensional gel electrophoresis, Western blotting, ELISA, protein or peptide mass fingerprinting (e.g., using MALDI-TOF/TOF), multi-dimensional electrophoresis (e.g., solution phase isoelectric focusing followed by two-dimensional gel electrophoresis of concentrated pI fractions), mass spectrometry (MALDI-MS, LC-MS/MS, MALDI-TOF MS, or LC-ESI-MS/MS), PCR, RT-PCR, microarrays, thin-layer chromatography, liquid chromatography, gas chromatography, GC/MS, electron microscopy, fluorescent microscopy, or surface analysis methods.

In some embodiments, the component is analyzed for the presence of a second component (e.g., a protein, an enzyme, a DNA sequence (e.g., a mutation, methylation, and other adduct), an RNA sequence (e.g., a mutation, maturation), a metabolite).

In some embodiments, the method further includes a purification step.

In some embodiments, the purification step includes a centrifugation step or a filtration step.

In some embodiments, the centrifugation step or the filtration step is performed before the extraction.

In some embodiments, the centrifugation step or the filtration step is performed after the extraction.

In some embodiments, the method includes an additional fractionation step.

In some embodiments, the fractionation step is performed is performed before the extraction.

In some embodiments, the fractionation step is performed after the extraction.

In some embodiments, the fractionation step includes centrifugation, chromatography including buoyant density accumulation (BDA), sucrose gradient separation, HPLC, affinity binding chromatography, SEC, electrophoresis, filtration, or dialysis.

As used herein, the term “extracting” refers to obtaining (e.g., isolating) a component of interest from a source of the component (e.g., from a sample, e.g., a biological sample). For example, extracting a molecular complex, e.g., from a source, removes the molecular complex from at least about 10%, about 20%, about 30%, about 40%, about 50%, about 60%, about 70%, about 80%, about 90%, about 95%, or about 99% of the other materials or components that were present prior to the extracting, e.g., in the source of the component. Methods of extracting are also referred to as methods of extraction.

The term “fractionation,” as used herein, refers to the separation of a mixture into at least two components, for example, by distillation, chromatography, centrifugation, filtration or crystallization. However, when separation of distinct molecular complexes is intended, the goal is to retain the structural and, in some embodiments, functional integrity of some complexes (e.g., complexes of interest), while disassembling, disrupting, dissolving or otherwise removing other molecular complexes contained in a given biological sample. In some cases, if separation of large complex structures, e.g., intact subcellular organelles surrounded by a lipid bilayer is intended, differentiation of the organelles by the properties of their surface (antibody pull-down, immobilization on a specific surface, interaction with another complex, etc) or their size (ultracentrifugation in a density gradient or differential centrifugation) may not lead to a homogeneous fraction of a desired type of molecular complex due to the interference from fragments of molecular complexes or other components of the sample, i.e., the methods can result in a heterogeneous fraction instead. Addition of an orthogonal method (e.g., pressure cycling) to differentiate between distinct types of molecular complexes contained in a mixture (e.g., differentiation of distinct Gram-negative bacterial strains by the rigidity of their cell wall or differentiation of two or more types of mammalian cells in a block of tissue by their viability after their exposure to altered hydrostatic pressure) results in an enhanced ability to enrich for a particular molecular complex while inactivating, disassembling or dissolving other types of molecular complexes.

In some aspects, the disclosure provides a method of extracting an organelle complex from a sample. The method includes providing a mixture at a first pressure, P₀, the mixture containing a sample (solid or liquid) and an aqueous or liquid phase that contains a buffer; subjecting the mixture to an elevated pressure, P₁, of from about 1,000 psi to about 60,000 psi for at least about 1-2 seconds; subjecting the mixture to a pressure P₂, wherein P₂ is less than P₁; and fractionating the mixture thereby extracting a molecular complex from the sample.

In some aspects, the disclosure provides a method of extracting mitochondria from a sample (e.g., a tissue or cell suspension). The method includes providing a mixture which is subjected to a first pressure, P₀, which is usually atmospheric pressure. The mixture contains a sample (e.g., tissue or cell suspension) and an aqueous mixture, the aqueous mixture contains a buffer. The mixture is subjected to an elevated pressure, P₁, of from about 1,000 psi to about 60,000 psi, for at least about 1-5 seconds; subjecting the mixture to a pressure P₂, wherein P₂ is less than P₁; and fractionating the mixture thereby extracting a molecular complex (e.g., mitochondria) from the sample.

A method for extracting molecular complexes using cyclic hydrostatic pressure is described. A typical biological sample can be, e.g., in liquid, suspension, semi-solid, or solid form. For example, a microbe can be found in a culture solution. Tissue may be found in a fragment or whole piece. Because of the diversity and complexity of biological samples, a broadly applicable sample preparation method was developed for many types and forms of biological samples. This described pressure-based extraction method has the potential and feasibility of processing a wide variety of biological samples.

As one example, samples can be put into a sample processing container, e.g., a PULSE™ Tube (Pressure Biosciences, Inc.). PULSE™ Tubes (Pressure Used to Lyse Samples for Extraction) transmit the power of pressure-based cycling from the BAROCYCLER™ instruments (Pressure Biosciences, Inc.) to the sample. Briefly, specimens are placed inside the PULSE™ Tube, the PULSE™ tube is placed in the pressure chamber, pressure chamber fluid is transported, compressed and delivered by the pressurization equipment, e.g., BAROCYCLER™, and pressurization begins. As pressure increases, the ram pushes the sample from the sample chamber through the lysis disk and into the fluid retention chamber. Cells in suspension can be loaded from the cap side. (In the cases when the sample is particularly hard and rigid, e.g. bone or teeth, sample can be loaded from the cap or the buffer loading end, and the sample is not directly pushed or compressed by the ram). When pressure is released, some of the sample (now mostly or partially homogenized) is pulled back through the lysis disk by the receding ram. The combination of physical passage through the lysis disk, rapid pressure changes, and other accompanying biophysical mechanisms, break up the cellular structures quickly and efficiently, releasing subcellular components, e.g., organelles, molecular complexes, e.g. protein-nucleic acid complexes, small molecules and protein complexes, and multimeric protein complexes.

Sample containers for the pressure process can be made in a variety of forms and shapes. The PULSE™ tube has the capability to hold either liquid or solid forms of biological samples during storage and processing under hydrostatic pressure. Other forms of sample containers may also be used. For example, a sealed plastic pouch can be used to hold the sample and be processed under pressure. In some cases, modifications in the basic PULSE™ Tube can be supplemented with additional features to improve the efficacy of the process. For example, 0.5 mm silica or glass beads can be added to assist the extraction process, where beads are introduced, e.g., in 1:1 or 10:1, or 100:1 bead to sample volume ratio. The presence of the beads has been found beneficial, e.g., when processing samples that have a cell wall, cartilage or polysaccharides. In some embodiments, samples are introduced in the PULSE™ Tube from the cap end (although in most cases, samples are introduced from the ram end). This includes examples such as bone, tail, hard wood, and seed. In some cases, the sample is packaged in the sample container after brief mincing or breaking, e.g., with a grinder, or scissors, or mortar and pestle.

The hydrostatic pressure can be hydraulic or pneumatic pressure transmitted to the sample via compressed gas or air, liquid, or solid. When an air pump is used to generate high pressure, pressure is delivered, e.g., through a piston and via a pressure fluid. When liquid or solid media are used for pressurization of the sample(s), the sample container may be in direct contact with the liquid or solid. Depending on the sample size, the pressure device can accommodate samples as large as liter volumes, or as small as microliters, or even in sub-microliter scales. The sample size can be, e.g., animal tissues, or plant materials between milligrams and kilograms, or, e.g., biological fluid, or a sample solution in 10 microliters to hundreds of liters. In some embodiments, sample sizes in microliters or sub-microliters are processed. A sample size can be, e.g., in milligrams, e.g., 1 to 400 mg. The upper limit of sample size may be restricted by the size of the sample container, or the pressure chamber. It can be advantageous if the ratio between sample and extraction buffer is greater than 1:2, e.g., 1:5, or 1:10, or 1:20, which would provide better solvent exposure area so that improved extraction efficiency may be achieved. In some embodiments, samples sizes of about 10 microliters to about 50 ml are used.

The extracted molecular complexes can be used in several biologically related applications, e.g., for analytical discoveries, diagnostics, preparation of products, and/or drug discoveries. For example, this process can facilitate the enrichment for proteins (or biomarkers) localized in organelles. For example, with some proteomic techniques, whole cells or tissues are homogenized for protein and proteome isolations, which may lead to a higher level of complexity of these proteomes. Only certain tissues, such as eye, have a limited number of proteins in the proteome, for which the whole tissue protein extraction may be feasible. For other types of tissues, due to the complexity in their proteome and limitations in the analytical capabilities of available proteomic analytical methods, molecular complex (e.g., organelle) extraction from tissues or cells can be crucial, because the molecular complexes (e.g., organelle proteome) have significantly fewer proteins than the whole cells or tissues. By employing this novel method, one could take advantage of the simplified biological machinery of molecular complexes, such as organelles, to study not only the components of the subcellular complex proteomes, but also the function and biochemical reactions of these molecular complexes, e.g. organelles. An example is provided herein—one can extract mitochondria and follow the extraction/isolation by mitochondrial DNA purification or by Western blotting for mitochondria-specific proteins. This method can be employed, e.g., when mitochondrial DNA, but not genomic DNA, is required, or when intact purified mitochondria are required. It can also be employed for the purification or enrichment of certain organelles or molecular complexes. For example, one may use the method to enrich for lysosomes, channel protein complexes, and ribosomes. In addition, one may enrich for nuclei in order to extract DNA-transcription factor complexes for analysis by chromatin immunoprecipitation (ChIP), or other methods. Moreover, membrane vesicles, sometimes termed microsomes can be made from larger membrane structures of the cell such as endoplasmic reticulum (ER) or Golgi complex.

Sample sizes for use with the methods described herein can be, e.g., in microgram, microliter, kilogram(s) or up to thousand-liter amounts as described above. The sample can be, e.g., biopsy tissues or materials from animal and plant, microbe in hosting matrices or in cultures, cell cultures, samples that are not biological, but contaminated with biological materials, or a preparation of molecular complexes from an artificial organism.

The samples that can be processed include, e.g., bacteria, cultured cells, insects, fungi, plant tissue, animal tissue, plant or animal tissue infected with microorganisms, e.g. bacteria, fungi, and virus, raw material containing biological specimen, archaeological or paleontological specimens, and so forth.

The method is applicable, e.g., to frozen, fresh, chemical-fixed, and/or ancient samples.

This method may be carried out, e.g., in a clinical, research, industrial, military, forensic, and educational laboratories, both stationary and mobile.

All herein cited patents, patent applications, and references are hereby incorporated by reference in their entireties. In the case of conflict, the present application controls.

The details of one or more embodiments of the invention are set forth in the accompanying drawings and the descriptions below. Other features, objects, and advantages of the invention will be apparent from the description and drawings, and from the claims.

### DESCRIPTION OF DRAWINGS

FIG. 1 is a diagram illustrating a pressure cycle.

FIG. 2 is an illustration of protein patterns obtained by separation of cell lysates on polyacrylamide gels, showing advantages of alternating hydrostatic pressure for cell lysis. Labels show processing conditions.

FIG. 3 is a graph depicting total mDNA yields at different fixed pressures with increasing cycles.

FIG. 4 is a graph depicting total mDNA yields with different numbers of cycles and different levels of pressure.

FIG. 5 is a graph depicting total mitochondrial DNA yields at different centrifuge speeds.

FIGS. 6A and 6B are graphs depicting the correlation of mitochondria yields and amount of tissue processed by pressure cycling. In FIG. 6A, total yield of mDNA versus total sample weight is shown. In FIG. 6B, mitochondria yields normalized by weight (number of mDNA per 10 mg of loaded sample).

FIGS. 7A and 7B are graphs depicting mDNA yield and efficiency of mitochondria isolation. FIG. 7A is a graph depicting the mDNA yield versus round number of pressure cycling. FIG. 7B is a graph depicting the percent efficiency of repeated pressure cycling processing compared to the Dounce homogenizer method (100%).

FIG. 8 is a bar graph depicting the effect of different buffers on mitochondria yields. 1: PBS; 2: HEPES+5 mM MgCl2 1 mM EDTA pH 7.3; 3: HEPES+5 mM MgCl2+1 mM EDTA+250 mM Sucrose, pH 7.3; 4: processing in a Dounce homogenizer in PBS.

FIGS. 9A and 9B are bar graphs depicting the effect of pressure and the addition of glass beads on recovery of mitochondrial DNA from rat cardiac tissue. FIG. shows pressure profiles of mitochondrial DNA recovery from rat heart tissue. 200 mg of rat heart tissue were processed under the different pressure in PBS with ten (10) cycles of 20 sec high and 10 sec ambient pressure, 4° C. CytB copy numbers from isolated mitochondria were quantitatively determined using real-time PCR. FIG. 9 B shows that, in the presence of glass beads (0.5 mm or 1.0 mm in diameter), larger numbers of mitochondria were obtained than without beads (none, shown as the first data bar in the graph). Beads were in roughly equivalent volume as the sample and loaded from the Ram side of the PULSE™ Tube along with the sample. Ten cycles of pressure between ambient (10 sec) and 20 kpsi (20 sec) were applied to the samples in PBS at 4° C.

FIGS. 10A and 10B are bar graphs depicting the influence of pressure and the number of cycles on mitochondrial cytochrome c oxidase activity. FIG. 10A is a bar graph depicting the influence of the number of pressure cycles on mitochondrial cytochrome C oxidase activity. FIG. 10B is a bar graph depicting the influence of cycling at various levels of pressure on mitochondria cytochrome c oxidase activity.

FIG. 11 is a bar graph depicting mitochondrial outer membrane integrity using pressure cycling at different pressure levels.

FIG. 12 is a bar graph depicting the influence of the number of pressure cycles on mitochondrial outer membrane integrity.

FIG. 13 is a bar graph showing a comparison of Cytochrome c oxidase activity in samples processed by pressure cycling or Dounce homogenizer.

FIGS. 14A and 14B are illustrations depicting enrichment of mitochondrial fractions from cultured cells as a function of pressure and the number of cycles. FIG. 14A presents Western blots of pellet 1, 2 and supernatant. The top blot in FIG. 14A was probed using an anti-GAPDH antibody (1:200 mouse monoclonal) as a loading control. The bottom blot was probed with anti-VDAC/Porin which is a mitochondrial marker (1:500 Rabbit polyclonal). FIG. 14B presents Western blots and a Coomassie blue-stained gel of pellet 1, 2, supernatant and wash fractions from PC12 cells. VDAC/Porin is a marker of the mitochondrial outer membrane, Prohibitin is a marker of the mitochondrial inner membrane and HSP60 is a marker of the mitochondrial inner matrix space. The presence of all 3 proteins in the mitochondrial fraction (pellet 2) supports the conclusion that this fraction contains intact mitochondria.

FIG. 15 is an illustration of the mitochondrial extraction from various rat tissues.

FIGS. 16A and 16B are illustrations depicting electron micrographs of isolated mitochondria obtained by various methods. FIG. 16A: Mitochondria extracted by Dounce homogenizer method; FIG. 16B: Mitochondria from pressure cycling extraction.

FIG. 17 is an illustration depicting the differential effects of pressure at various stages of growth.

FIG. 18 is a graph showing the effects of pressure on viability and integrity of nematodes C. elegans. The effects of pressure on disruption of C. elegans. At 20,000 psi, 2.2% of the nematodes survive. At 30,000, 100% are killed with only viable embryos remaining. Synchronized cultures have been produced from 30 and 40 kpsi pellets.

FIGS. 19A and 19B are illustrations depicting electron micrographs of isolated mitochondria obtained by various methods. FIG. 19A: Mitochondria extracted by pressure cycling; FIG. 19B: Mitochondria extracted by using a standard homogenizer.

FIG. 20 is a line graph showing the survival levels of P19 embryonal carcinoma cells subjected to pressure cycling at different pressures.

FIG. 21 is a series of four panels showing P19 embryonal cells at day 4, 10, 14, and 21 after replating after 10 cycles at 25,000 psi.

FIGS. 22A-22K are a series of panels showing control or pressure-treated P19 embryonal cells after replating. Control cells: panels A, B, C, D; pressure-treated cells: panels E, F, G, H, I, J, K.

### DETAILED DESCRIPTION

### Overview

This disclosure describes the extraction of molecular complexes using alternating hydrostatic pressure, e.g., so that the complexes can be produced (e.g., isolated), e.g., for genomic research, proteomic research and/or biological functional studies. Because pressure is a physical force and can be precisely regulated (e.g., using an instrument), along with the appropriate temperature and buffer conditions, and other physical or chemical variants, e.g. light, sound wave, magnetic field, nanomaterials, this pressure-based extraction method, which includes cycles of high hydrostatic pressure, may be utilized as a highly precise, effective, differential, and reliable solution in sample preparation.

One consideration in sample preparation is the production of extracts in which cellular and molecular complexes are preserved and ready for in vitro studies. This is of particular interest, e.g., in fundamental biological studies and searches for therapeutic agents or drugs. For example, proteomes of whole cells or tissues can be highly complicated and difficult to dissect. However, the proteome may be greatly simplified by focusing on specifically-isolated molecular complexes. Another application for extracted molecular complexes is in the study of in vitro biochemical properties of these extracted and/or isolated complexes. Certain methods for the extraction of biomolecules from cells and tissues can lack the desired specificity and/or may be too damaging to facilitate extraction of molecular complexes with maximum yield. These methods may rely on either the use of aggressive chemicals or the use of vigorous physical shearing forces to disrupt samples (e.g., to break cells open). For example, bead milling, sonication, and rotor-stator homogenization are common mechanical tools for extraction. These procedures may damage molecular covalent bonds by heating, shearing or activating endogenous enzymatic hydrolysis, and/or by protein denaturation, which may cause dysfunction or damage to the molecular complex of interest. There continues to be a need for well-controlled, automated sample extraction systems that not only simplify the extraction process, but also satisfy diverse but important requirements in maintaining the structure and/or function of the molecular complexes of interest. An automated system in which physical and chemical parameters can be well-controlled is highly desirable. It is also advantageous for the process to be applicable to as wide a range of distinct samples and target types as possible.

Moreover, selectivity and yield of extraction can be significantly improved if a combination of orthogonal physical and/or chemical treatments (i.e., methods employing distinctly different physical of chemical phenomena or exploring distinct differences in physiochemical properties between sample components to be separated) is employed to selectively preserve a desired type of molecular complex and degrade or separate other types, leading to a possible and feasible homogenous fraction of the molecular complex.

A variety of techniques are available for preparing protein and nucleic acid extracts for genomics and proteomics. To provide accurate representation of the proteomes for proteomic analysis, pre-analytical extraction techniques are needed to extract targeted cellular components, such as compartmental proteins or organelles, in relatively large quantities.

Pressure cycling-based extraction methods exhibit unique orthogonal features compared to conventional methods. For example, as pressure is applied to a sample in solution, the disruption of protein structures, such as cellular membranes, breaks open the cells, allowing for the release their contents. As pressure is released, the proteins resume their native configuration, but already disrupted cell membranes may not re-form. Repetitive applications of very short pulses of high pressure have been shown to be more effective in releasing cellular contents than one continuous pressure pulse. More significantly, the biological activity of enzymes released by pressure cycling retains much greater function than the activity of enzymes obtained by continuous pressure or by the use of other physical means or chemical-based processes. Pressure cycling-based methods release at least as many proteins as other current extraction methods. Further, in several cases, distinct protein species were found in the pressure-cycling extracts, in particular, high molecular weight species, and hydrophobic proteins and molecular complexes. Further discoveries may be possible using pressure cycling for the extraction of proteins that may not be possible with other extraction methods. Because the pressure-cycling method is instrument-based, it has excellent potential to be developed as a high precision extraction system.

Larger objects enclosed into membranes are typically more susceptible to disruption during pressure cycling than smaller or organelles or protein complexes, e.g., nuclei, mitochondria, ribosomes, etc. This effect can be explained, at least in part, by several phenomena. First, overall compressibility of any membrane-enclosed object is proportional to its original volume. Reduction of volume during compression is expected to cause greater structural changes in a membrane of a larger object which would lead to destabilization of intra-molecular interactions in and disruption of the lipid bilayer. Second, several sub-cellular organelles, e.g., nuclei or mitochondria, are surrounded by two layers of membrane. Such organelles tend to be more structurally stable under hydrostatic pressure treatment.

In addition to temperature, physical factors, e.g., volume and geometry of the sample container, physical contacts of the sample with various features of the sample container, the extent of physical force applied to the sample during treatment, and/or the duration of the treatment can contribute to raising the extraction efficiency and, therefore, resulting in higher yields of the purified or enriched target. In some embodiments, the sample is soft to allow gas and liquid extraction media (e.g., buffer) to penetrate into the interior of the sample. The extraction efficiency may also be enhanced by optimization of the extraction media volume to match the available sample size by addition of glass beads, mineral oil or similar chemically inert solid or liquid materials together with the sample during the pressure cycling process. Further, the sample may be dissected into a size and shape suitable for the appropriate sample container and into geometry more favorable to extraction.

Mitochondria

Biological research and clinical diagnostics that analyze organelles and molecular complexes have demonstrated that certain subcellular structures, such as mitochondria, endoplasmic reticulum, and channels in the form of membrane proteins, play important roles in the regulation and progression of diseases such as cancer, diabetes, obesity, and other metabolic disorders. For example, mitochondria play key roles in many diseases and in aging. About one in 4,000 children in the United States will develop a mitochondrial disease by the age of 10 years. One thousand to 4,000 children per year in the United States are born with some type of mitochondrial disease. In adults, many diseases of aging have been found to be associated with defects in mitochondrial function. Defects in mitochondrial function have now been linked to many of the most common diseases of the aged population. These include type II diabetes mellitus, Parkinson Disease, atherosclerotic heart disease, stroke, Alzheimer dementia, and cancer. It is possible that mitochondrial impairment might be at the heart of many more diseases and disorders. This impairment results in high levels of free radicals that not only continually damage the mitochondria, but other important parts of the cell (e.g., DNA), leading to a decrease in overall cell function. Mitochondrial decay may also result in energy deficits and an inability to dispose of toxins from the environment, and may cause cells to die prematurely. Mitochondria play an important role in apoptosis, a fundamental biological process by which cells die in a well-controlled or programmed manner. A number of genetic studies have implicated a role for mutational hotspots in the mitochondrial genome which are associated with development of ovarian or colon cancer. Additional studies have identified mitochondrial markers that may be of clinical significance in hepatic, esophageal, pancreatic, prostate, brain, and other cancers. However, the true significance of such biomarkers can only be validated using qualitative and quantitative protein studies, which will provide the identification of these markers and their interaction with intracellular structures, such as the mitochondria. The field of mitochondrial research is currently among the fastest growing disciplines in biomedicine. To facilitate rapid data generation, instruments and procedures are needed for isolation of molecular complexes in cancer research and clinical diagnostics. One extremely important prerequisite for these studies is appropriate sample preparation of molecular complexes, e.g. organelles and compartmental proteins.

Pressure

Hydrostatic pressure (e.g., pressure cycling, including one cycle of pressure manipulation) can be included in a method to isolate molecular complexes in a combination of other physical and chemical variants. The susceptibility of a given molecular complex to disruption by pressure (e.g., pressure cycling) depends upon the physical properties of the complex, e.g., the compositions of protein(s) in the complex, the lipid compositions of the membrane, the presence/absence of a cell wall, the heterogeneity of a bilayer, the heterogeneous distribution of various lipid molecules in the bilayer, air content surrounding the lipid bilayer, physical-chemical properties of the environment, e.g. temperature and the buffer conditions being used. The physical properties of the molecular complex of interest may exhibit distinctive profiles of the tolerance to osmotic pressure, and to hydrostatic pressure at specific conditions. Because of these differing properties or factors, alternating or cycling in pressure can lead to the disruption of certain complexes while other complexes remain intact, allowing the isolation of the intact complex.

The pressure can be applied e.g., as hydraulic or pneumatic pressure. The hydraulic pressure is transmitted through a pressure medium. This medium is selected based on certain conditions, e.g. equipment design and operating temperature requirements. Water, deionized water and water with an antifreeze mixture, e.g., ethylene glycol, and propylene glycol, can be used as the pressure medium.

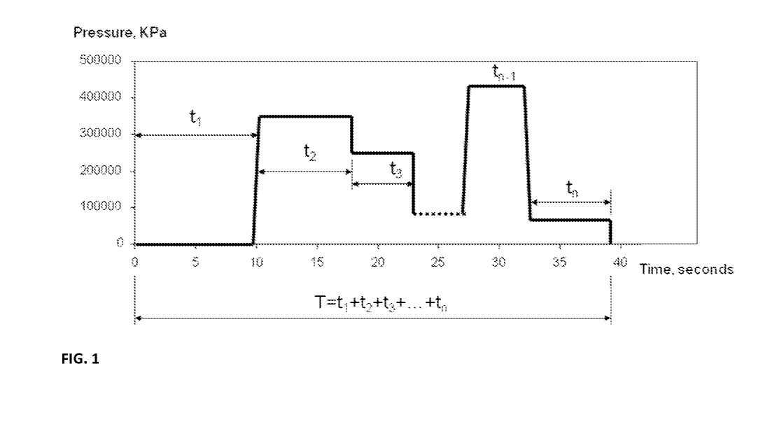

A pressure cycle is the summation of exposing a sample to more than one pressure for a period of time at each pressure level. For example, a pressure cycle can consist of exposing a sample (e.g., the mixture being exposed to pressure cycles, e.g., the mixture containing a component of interest) to a first pressure for a first period of time and exposing a sample to a second pressure for a second period of time. However, there is no limit to the number of pressures that the sample needs to be exposed to, and the period of time spent at each pressure does not have to be the same. For example, as illustrated in FIG. 1, a sample is exposed to a first pressure for a period of time (t₁). The sample is then exposed to a second pressure for a period of time (t₂). The sample is then exposed to a third pressure for a period of time (t₃). The sample can be exposed to various pressures for various periods of time (tₙ). The summation of these exposures to each pressure for each period of time is a pressure cycle. In some embodiments, the sample is exposed to a pressure that is greater than the first or second pressures for a period of time (illustrated as tₙ₋₁, in FIG. 1). Exposure to this pressure can, for example, introduce a reagent(s) into the mixture being exposed to the pressure cycles or a chamber (e.g., the chamber containing the mixture that is being exposed to pressure cycles) by rupturing a secondary container containing such reagent.

The maximum pressure to be used can be between about 1,000 psi and about 60,000 psi, e.g., between about 2,000 psi and about 40,000 psi, between about 3,000 psi and about 20,000 psi, between about 2,500 and about 15,000 psi, between about 4,000 psi and about 12,000 psi, and preferably about 5,000 psi to about 10,000 psi.

The preferred upper ranges of pressure can be optimized for the targeted molecular complex, e.g., pressures that correspond to the results as shown in the examples using mitochondria. For example, in some experiments, mitochondria were extracted from rat liver using 5 cycles of 20 sec at high pressure of 5 kpsi, and 10 sec ambient pressure at 4° C. When mitochondria are extracted from rat heart, pressure cycles at 20 kpsi are more effective. Each kind of molecular complex may have unique pressure sensitivity, which is determined by its own molecular characteristics, buffer, temperature, and other factors including the pressure profile applied in the extraction process. Thus, optimization studies may be necessary and variables can be optimized when a specific target has been selected. The pressure profiles may also need to be optimized depending on the pressure instrument and sample container. It is also possible that different molecular complexes may exhibit similar pressure profiles in certain cases. In these cases, it may be difficult to selectively disrupt one type of molecular complex and not the other using pressure. However, the different complexes can be separated by choosing appropriate fractionation protocols, such as differential centrifugation, antibody affinity chromatography or size exclusion chromatography. In some case, one may disrupt or strengthen one type of complex as opposed to the others by doping with appropriate chemicals, such as, phospholipids, proteases, or chelating agents.

The minimum pressure to be used can be between about 1 atm (14.7 psi) to about 15 kpsi. In some embodiments, the minimum pressure used is the pressure at deep-sea level, e.g., about 100 kPa e.g., 101.3 kPa or 15 kpsi. The initial starting pressure (P₀), is often atmospheric pressure. In some embodiments, special sample tubes may be designed so that the initial processing or the minimum pressure applied to the sample is above the atmospheric pressure, e.g., up to 15 kpsi for organisms found in deep sea environments. This operation can be applied to the extraction of organelles or other molecular complexes that are less stable at atmospheric pressure, for example, organisms that are found in deep-sea or piezophiles (species normally grown under pressure).

In some embodiments, the maximum and minimum pressures chosen are based on providing a minimum or maximum difference in pressure values. For example, the minimum and maximum pressures differ by no more than about 200 MPa. As another example, the minimum and maximum pressures differ by no less than about 100 kPa.

In some embodiments, the rate of change from the maximum to the minimum pressure is between about 1 and about 1,000 psi/millisecond. The preferred rate of change between pressure levels can be a fraction of a second (e.g., as fast as when a valve is opened) or as long as about 1-2 seconds. The rate at which pressure is increased is typically determined by a pressure generator or a pump. The interior volume of a pressure chamber, the compressibility of the pressure medium, and the compressibility of samples also play roles in the rate of pressure increases. The rate of pressure going down (e.g., decompression) can be important, as oscillation of micro-bubbles created when pressure is suddenly decreased may contribute significantly to the disruption of relatively large cellular structures, e.g., cell membranes. The dynamic range of the disruptive pressure force caused by pressure cycling is proportional to the level of pressure. According to one model, decompression may be more important than compression in an extraction and in the disruption of cellular structures. In general, a more rapid drop in pressure can more disruptive and therefore more effective than a comparatively less rapid decrease in pressure.

The number of pressure cycles (e.g., the number of times the pressure is changed from a first value to a second value or the number of times the pressure changes) used is also a factor that affects the extraction. For example, the number of pressure cycles can range between about 1 cycle to about 250 cycles, e.g., from about 5 cycles to about 225 cycles, from about 10 cycles to about 200 cycles, from about 20 cycles to about 150 cycles, from about 30 cycles to about 100 cycles, from about 50 cycles to about 80 cycles, from about 100 to about 300 cycles, from about 200 to about 400 cycles, from about 50 to about 150 cycles, from about 5 to about 35 cycles, from about 10 to about 25 cycles. The number of cycles can depend on a number of factors, such as the pressure profiles of each cycle, and extraction reactions in releasing targeted molecular complexes, including physical and chemical factors in additional to pressure. For example, the number of cycles can be between about 1 and about 250. In some embodiments, 5 cycles of pressure can produce 20 to 60% release of targeted molecular complex, such as mitochondria or nuclei from liver tissues.

In some embodiments, the pressure cycles from a first pressure to a second pressure (e.g., that is higher than the first pressure) to a third pressure (e.g., that is lower than the second pressure; the third pressure may or may not be the same as the first pressure), and so on. In these embodiments, all three (or more) pressures are included as part of the cycle.

The length of the pressure cycles (the total amount of time spent in the cycle, i.e., the amount of time spent at the first pressure plus the amount of time spent at the second pressure, plus the amount of time spent at any additional pressure(s) (e.g., at a third pressure, a fourth pressure, etc.)) is also important. For example, the length of the cycle may be from about 5 seconds to about 60 minutes, e.g., about 10 seconds, about 20 seconds, about 30 seconds, about 45 seconds, about 60 seconds, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 15 minutes, about 20 minutes, about 25 minutes, about 30 minutes, about 35 minutes, about 40 minutes, about 45 minutes, about 50 minutes, about 55 minutes, about 60 minutes. In many embodiments, the length of time at the first and second pressures is the same. For example, in a 20 second cycle, the mixture is at the first pressure for 10 seconds and at the second pressure for 10 seconds.

The length of time spent at a given pressure level (e.g., at the first or second or third pressure) can be, e.g., from about 1 second to about 30 minutes, e.g., about 1 second, about 5 seconds, about 10 seconds, about 20 seconds, about 30 seconds, about 45 seconds, about 60 seconds, about 2 minutes, about 3 minutes, about 4 minutes, about 5 minutes about 6 minutes, about 7 minutes, about 8 minutes, about 9 minutes, about 10 minutes, about 11 minutes, about 12 minutes, about 15 minutes, about 20 minutes, about 25 minutes, about 30 minutes. In many embodiments, the length of time at the first and second pressures is the same. For example, in a 20 second cycle, the mixture is at the first pressure for 10 seconds and at the second pressure for 10 seconds.

The exposure to a particular pressure level may need to be optimized based on the properties of the solvents and composition of the plurality of components. Thus, the length of time spent at one pressure may need to be longer than the time spent at the other pressure(s). In some embodiments, the mixture may be at each pressure for a different amount of time. For example, the mixture can be at the first pressure for 10 seconds and at the second pressure for 30 seconds.

Further examples of pressure cycles are as follows:

Start at atmospheric pressure, e.g. at sea level (101.3 KPa), followed by 50 MPa held for 5 seconds and 10 seconds held at atmospheric pressure, 5 cycles;

Start at atmospheric pressure, followed by 5 seconds at 150 MPa and 10 seconds at atmospheric pressure at sea level (101.3 KPa), 20 cycles;

Start at 100 MPa, followed by 250 MPa held for 10 seconds followed by 200 MPa held for 5 seconds followed by 100 MPa held for 5 seconds, the sequence repeated for 10 cycles.

In some embodiments involving three pressures in the cycle, the length of the pressure cycle is the total amount of time spent at the first, second, and third pressures.

Additional examples of pressure cycling parameters include: five cycles at 5 kpsi, where pressure is kept for 10 seconds at 5 kpsi, followed by 20 seconds at approximately 14.7 psi (atmospheric pressure); 20 cycles where a pressure of 15 kpsi or 100 MPa is held for 5 seconds and atmospheric pressure (14.7 psi or 101.3 kPa) is held for 20 seconds during each cycle; 30 cycles where pressure is maintained at 22 kpsi or 150 MPa for 10 seconds, followed by the step at 5 kpsi or 34 MPa for 20 seconds, which is then followed by 30 seconds at 15 kpsi or 100 MPa, resulting in a 1 minute pressure cycle.

Temperature

The temperature at which the extraction methods are performed can also influence the process. Temperature can increase the fluidity of cellular components in samples (e.g., biological membranes) and can contribute to the extraction of a molecular complex of interest. Temperature fluctuation experienced by the sample during the pressure process may be influenced by the sample properties, pressure chamber thermo-conductivity, pressure changing rates, and duration of incubation at each pressure level. It can be related to an individual pressure generator, a pressure medium or fluid, heat-transfer properties of the pressure chamber, and the heat capacity of the circulating medium or fluid which is used to control the chamber temperature.

For example, the extraction methods can be performed at a temperature from about 0° C. to about +100° C., e.g., from about 0° C. to about 70° C., from about 0° C. to about 50° C., from 4° C. to about 37° C., from about 10° C. to about 30° C., from about 15° C. to about 25° C., at about 20° C., at about 23° C., at about 25° C., or at about 70° C. In some embodiments, the temperature is between about 4° C. and about 37° C. The temperature can be higher, e.g., above 37° C., e.g., about 95° C., e.g., if the sample is from a thermophilic species and/or the molecular complex of interest is stable at temperatures above about 37° C. In some embodiments, the methods can be performed, e.g., at temperatures below about 0° C., e.g., between about −30° C. and about 0° C., e.g., at about −4° C., about −10° C., or about −30° C.

The choice of temperature(s) for use in the methods can be influenced by the properties of the sample components (e.g., the cells, tissues, and/or complexes (e.g., molecular complexes, e.g., a complex of interest)). The temperature can be optimized by altering (increasing or decreasing) the temperature in 1° C. increments. The temperature at which the method is carried out can be regulated, e.g., by a circulating water bath, a fan or air source that provides ambient, hot or cold airflow, or a solid state Peltier temperature controller.

The extraction methods can also be carried out such that the temperature and the pressure vary within each cycle, since temperature changes can further alter the physical properties of molecular complexes and/or other sample components. For example, at the first pressure in the cycle, the sample (mixture) is at a first temperature; at the second pressure of the cycle, the sample (mixture) is at a second temperature. In some embodiments, the first temperature is higher than the second temperature. In other embodiments, the second temperature is higher than the first temperature.

Liquids

A variety of liquids can be used in the extractions methods provided herein. For example, solvents, detergents, buffers, chaotropic agents (e.g., chaotropic salts), and mixtures thereof can be used. For example, the extraction buffers may contain one or more of: an aqueous solution with salt, pH buffer, electrolytes, enzymes and osmotic pressure buffer components. At least one, and sometimes more than one, buffer(s) is utilized for the extraction. The different components of buffers are chosen and optimized for selective release of certain types of molecular complexes. For example, the pressure treatment used in the extraction methods described herein may be a repetitive application of multiple sets of pressure treatment, and buffers can be exchanged and fresh buffers can be applied to the sample being subjected to the extraction. For example, the sample being processed is exposed to several loads of fresh extraction buffers. By replacing buffers, the extraction efficiency can be significantly improved as compared to the efficiency when one load of buffer is used with the same number of pressure cycles.

Solvents

Aqueous solution or water with soluble buffer components is the primary useful solvent in organelle extraction. In some cases, mixtures of solvents can also be employed in the extraction methods described herein. The solvents used in the extraction methods are often aqueous and miscible. For example, water with miscible organic solvents, e.g. acetone, acetonitrile, ethanol, 1-butanol, 2-butanol, 2-butanone, t-butyl alcohol, 1-propanol, 2-propanol, diethylene glycol, hexafluoroisopropanol (HFIP), dimethyl sulfoxide (DMSO), ethylene glycol, glycerin, methanol, heavy water (D₂O), and mixtures thereof. The solvent used can be formulated for releasing the targeted molecular complex from samples. Solvents can also be classified as protic or aprotic. Examples of protic solvents include water, methanol, ethanol, formic acid, hydrogen fluoride, and ammonia. Examples of aprotic solvents include dimethyl sulfoxide, dimethylformamide, hexamethylphosphorotriamide, and mixtures thereof.

Mixtures of any of the solvents described herein can also be used.

Non-limiting examples of solvents useful for practicing the methods of this disclosure include methanol, isopropanol, ethanol, water, acetonitrile, formic acid, trifluoroacetic acid, glycerol, a lipid (e.g., triglyceride, phospholipid, sphingolipid, glycolipids, oil, e.g., from sample itself, e.g., from a biological membrane (e.g., lipid membrane; lipid bilayer)), or aqueous solution (e.g., a liquid component(s) that originates from the sample itself, e.g., from a biological membrane or cytoplasm), a fluorocarbon, other halocarbon, dimethyl sulfoxide (DMSO), fluorinated alcohols (e.g., amphiphilic fluorinated alcohols) (e.g., 1,1,1,3,3,3-hexafluoro-2-propanol (HFIP), 2,2,2-trifluoroethanol (TFE), 2-fluoroethanol, 2,2,3,3-tetrafluoropropan-1-ol, 1,3-difluoropropan-2-ol, perfluorooctanol), perfluorooctanoic acid, other alcohols, and mixtures thereof.

In some embodiments, a sample (e.g., the source of components) provides (e.g., functions as) a solvent. In some cases, this solvent from the sample constitutes one of the liquids of the extraction system. When hydrophobic molecular complexes or affinity fraction materials are processed, micelles or gelatinous materials may be employed for the extraction. For example, when membrane proteins are isolated, Coomassie dyes can be introduced to induce an ionization change on the proteins and aminocaproic acid can serve to improve solubilization of membrane proteins.

The concentrations of the solvent(s) can be varied and optimized. Examples of concentrations include: about 0.2M HFIP; about 0.05M HFIP; about 0.38M to about 0.57M HFIP; about 60% HFIP; about 75% HFIP; about 95% HFIP; about 100% HFIP; about 1% to about 5% formic acid. The solvents can be made up in various other solvents (e.g., acetonitrile) or buffers (e.g., phosphate buffer or Tris buffer). The solvents can be used by themselves to constitute a phase in the methods described herein. Alternatively, a solvent (e.g., a solvent listed herein) can be a solvent that, along with another component (e.g., a liquid, e.g., another solvent) make up one solvent phase. A single solvent phase can include a combination of solvents. For example, a solvent phase can be acetonitrile:methanol:water in a 2:5:2 or 4:4:1 (v:v:v) ratio; or methanol:isopropanol in a 1:1 (v:v) ratio. As another example, 10% acetonitrile with 0.1% formic acid can be used as a solvent phase, as illustrated in the examples herein.

Buffers

A variety of buffers can be used with the extraction methods described herein. A wide variety of buffers can be used, e.g., to maintain a desired pH of an extraction solvent, to maintain osmotic pressure that is compatible with a molecular complex of interest, and/or to maintain compatibility with a subsequent analytical method. For example, PBS can be used in the methods. Additional buffers include HEPES, TRIS, MES, ammonium bicarbonate, formic acid, trifluoroacetic acid, acetic acid, a mitochondrial isolation buffer (MIB) (e.g., 250 mM mannitol, 0.5 mM EGTA, 5 mM HEPES, and 0.1% (w/v) BSA (pH 7.2) supplemented with the protease inhibitors of leupeptin (1 mg/ml), pepstatin A (1 mg/ml), antipain (50 mg/ml), and PMSF (0.1 mM); or 0.35 M sucrose, 10 mM Tris/HCl pH 7.5, 2 mM EDTA; or 210 mM mannitol, 60 mM sucrose, 10 mM KCl, 10 mM sodium succinate, 5 mM EGTA, 1 mM ADP, 0.5 mM DTT, and 20 mM Hepes-KOH, pH 7.5; or 200 mM mannitol/70 mM sucrose/1 mM EGTA/10 mM Hepes; pH 7.4), and so on.

The buffers used in the extraction methods are often aqueous and miscible. The buffer used can be formulated for releasing the targeted molecular complex from samples. The concentration of buffer components can be selected and optimized based on the biochemical and biophysical properties of the targeted molecular complex. The buffers can be composed of pH buffer components, osmotic pressure buffer components, and enzymes for removing certain contaminants. For example, a phosphate buffered saline (PBS, e.g., containing 137 mM NaCl, 2.7 mM KCl, 8 mM Na₂HPO₄, and 2 mM KH₂PO₄, pH 7.4) can be used when mitochondria are extracted from liver tissue. A HEPES buffer, containing 20 mM HEPES, 5 mM MgCl₂, 250 mM sucrose, 1 mM EDTA, pH 7.3, can also be used in mitochondrial extraction.

PBS buffer is often used in initial mitochondria DNA extraction. HEPES buffer with 250 mM sucrose is widely used in mitochondrial function studies.

PBS and HEPES buffer systems are more suitable for soft tissues in which cell walls, cartilage, polysaccharides, and/or cellulose are not present at high levels. When there are extracellular structures protecting cells from disruption, hydrolytic enzymes may be employed to facilitate and improve the access of extraction buffer and gas to the cells. An example is provided herein.

Various concentrations of salts, water soluble carbohydrates, e.g., sucrose, and other osmotically active reagents can be used to control osmotic pressure during the extraction of molecular complexes. For example, 0.9% sodium chloride (physiological saline solution) can be used in the extraction of various components from mammalian cells. Osmotic pressure can act synergistically with hydrostatic pressure in pressure cycling applications. For example, hypotonic concentrations of salts in the extraction solution can result in mammalian cell swelling due to the entry of water into the cell driven by the osmotic gradient. Such cell swelling can weaken the plasma membrane and can act synergistically with the pressure cycling to disrupt cellular plasma membranes. Conversely, isotonic salt concentrations can be used to protect, e.g., cells or organelles, e.g., from disruption at certain pressure cycling conditions, if such a result is desired. For example, for mammalian cells, sodium chloride (NaCl) concentrations below about 0.9% are hypotonic, and concentrations above about 0.9% are hypertonic. Plant cells resist swelling in a hypotonic environment due to the presence of a rigid cell wall. Instead, an excess hydrostatic pressure termed turgor pressure builds up within the plant cell, which may lower the effect of high hydrostatic pressure applied to the plant cell from the outside. Hypertonic media can cause shrinking of plant cells, which may enhance the effect of rapid de-pressurization on the disruption of the cell wall, and the associated plasma membrane, but preserve flexible organelle membranes from being ruptured.

Detergents and Chaotropic Agents

A detergent or a chaotropic agent (a.k.a., chaotropic salt) can be included in the methods to aid in the extraction of a molecular complex (e.g., organelle) of interest.

Examples of detergents that can be used include anionic detergents (e.g., SDS, Cholate, Deoxycholate); cationic detergents (e.g., C16TAB); amphoteric detergents (e.g., LysoPC, CHAPS, Zwittergent 3-14); and non-ionic detergents (e.g., Octylglucoside, Digitonin, C12E8, Lubrol, Triton X-100, Nonidet P-40, Tween 80). Several amphiphilic organic solvents, such as fluorinated alcohols (HFIP, TFE, perfluorooctanol, etc.) are frequently regarded as possessing detergent functionality. Such solvents can be used alone or in combination, as an additive to other solvents and buffer systems, e.g., solvent and buffer systems described herein.

The concentration of detergent can be, for example, from about 0.01% to about 10%, e.g., about 0.1% to about 2%, e.g., about 0.5% to about 4%, e.g., about 1% to about 2%.

A chaotropic agent can also be used. Examples of such agents include urea, guanidinium chloride, and guanidine hydrochloride. The concentration used can be about 0.01M to about 8M.

Multi-phase systems

Mixtures of aqueous solutions (buffers) and lipid compounds, which may or may not also contain surfactants, detergents, organic solvents, lipid bilayers, or liposomes can be used in extraction of certain molecular complexes, e.g., membrane proteins.

Other Components Present in the Liquids

The liquid phases described herein can optionally contain additional reagents. For example, an enzyme inhibitor, e.g., one or more of protease inhibitors (e.g., inhibitors of serine, cysteine, aspartic proteases or aminopeptidases) (e.g., 4-(2-aminoethyl)benzenesulfonyl fluoride (AEBSF), pepstatinA, E-64, bestatin, leupeptin, or aprotinin), DNAse inhibitors (e.g., aurintricarboxylic acid), RNAse inhibitors (e.g., SUPERASE•IN™ (Ambion), SCRIPTGUARD™ (Epicentre Biotechnologies), DEPC), metal chelating agents (e.g., DTPA, EDTA, EGTA, NTA, desferal) and so forth can be added to the liquid phases, e.g., to stabilize a component of interest, e.g., a molecular complex being extracted.

In some embodiments, the liquid phase can contain one or more enzymes, e.g., to aid in the extraction of a molecular complex of interest, e.g., the liquid phase may contain proteinase (e.g., a serine proteinase, e.g., nargarse, trypsin, chymotrypsin), DNAse, RNAse, lipase, and so forth.

As further examples, catalysts, enzyme solutions, inhibitors or substrates of enzymatic reactions can be introduced as a primary solution into the sample to initiate, modify or prevent a reaction which can be used to facilitate a desired effect on the cellular components or other molecular complexes for their extraction and isolation. For example, a substrate of an enzymatic reaction can be introduced into the sample containing the enzyme to initiate an enzymatic reaction. Such a reaction could produce a product used either to weaken certain undesired molecular complex(es) or stabilize desired molecular complexes for subsequent isolation. In other embodiments, an enzyme inhibitor can be introduced at a predefined time and/or hydrostatic pressure level as a secondary solution (e.g., from a secondary container) to facilitate inhibition of an enzymatic reaction, e.g., a hydrolytic enzyme capable of cell wall lysis can be present in an extraction solvent to weaken the cell wall surrounding the cells of single cell organisms or tissues. Once the desired degree of enzymatic digestion is achieved (e.g., the cell wall is weakened for subsequent disruption or destroyed entirely to produce cell protoplasts), the enzyme is inhibited by introduction of a suitable inhibitor via the release of respective secondary solution from a secondary container (e.g., a capsule) at a pre-defined pressure level and/or timing to protect intracellular components from being altered by the enzyme.

Secondary Containers

In some embodiments of the extraction methods described herein, in addition to the sample, one or more secondary container(s) (e.g., capsule, ampoule, plastic, latex or rubber seal) can be placed inside of the primary sample container and all of them subsequently are exposed to pressure cycles. The secondary container may contain a reagent or multiple reagents which will be introduced to the primary container during application of a certain level of pressure sufficient to cause the secondary container to release its contents (e.g., by rupture, by puncture, or by melting, and intrigued by light or sound waves). The reagent(s) in the second container, introduced during the application of pressure can lead to step-wise extraction functions, e.g., facilitate the extraction of sample components by step-wise destruction of sampled. For example, this reagent can serve as an additive to existing liquid(s) in the primary container. The second reagent(s) (e.g., the secondary solution) used in the extraction methods can be a supplement to the primary solution, e.g., to increase the stability of a component(s) of interest (e.g., molecular complex of interest) when pressure is increased to higher levels, to decrease the solubility of a component that is not of interest, and/or to increase the dissolution/solubility of a contaminant (e.g., a component that is not of interest). Examples of such reagents include inorganic (e.g., water based) solvents, amphyphilic solvents, solutions of chaotropic salts or detergents, detergent solutions (e.g., sodium dodecyl sulfate, [(3-Cholamidopropyl) dimethylammonio]-1-propanesulfonate, (CHAPS), Tween-80), organic solvents (e.g., hexane, pentane, methanol, ethanol, acetonitrile, methyl-tert-butyl ether (MTBE), n-propyl alcohol, isopropyl alcohol, isopentane, octane, decane, cyclohexane, xylene, benzene, toluene, etc.) or mixtures of thereof, and an amphiphilic reagent (e.g., HFIP, TFE).

Another example of the utility of reagent introduction into the sample chamber at a pre-defined time and/or pressure from a secondary container is the ability to qualitatively and quantitatively assay intracellular enzymes or other molecules when disruption (or dissociation) of a molecular complex of interest and supplements of the assay reagent(s) occur simultaneously. This application is particularly important when the molecular entities of interest are short-lived and are rapidly destroyed by chemical or enzymatic reactions once the integrity of the biological sample such as a cell, organelle, or other molecular complex is compromised during the extraction process. Also the assay reagent(s) are unstable, or short-lived, or poor-in-specificity in the primary extraction solution. The introduction of a specially selected molecular reporter ligand, e.g., specific chromogenic or fluorogenic substrate of enzymatic reaction, a chemical cross-linker reagent, an electrochemically active reporter molecule, or a chromogenic ligand possessing specific affinity to a molecule or molecular complex of interest, etc. can be introduced to quantify the presence of a chemically unstable molecular entity at the time of cell disruption.

One or more such reagents can be introduced, e.g., from the same or different secondary containers, e.g., upon pressure cycling to a certain pressure level. The secondary container can be designed to release its contents (e.g., rupture or leak) at or above a certain pressure level. In some embodiments, more than one secondary container can be used. For example, one secondary container can be designed to release its contents at one pressure, while a second secondary container can be designed to release its contents at a second pressure, and so forth. In this way, different reagents (or the same reagent in a separate dose) can be introduced into the mixture at controlled times (e.g., after a certain number of pressure cycles). The secondary containers are not limited in their shape or design. As used herein, the term “secondary container” refers to a sealed form whose contents include a reagent and that prevents the introduction of the reagent into the mixture or liquid phase contained in the secondary container until the pressure is raised to a level that causes the secondary container to release its contents. The material from which the secondary container is prepared is not limited. For example, the secondary container can be made of gelatinous material, lipid monolayer or bilayer, fragments of biological membrane, cellulosic polymers, glass, polymer (SAN, Polycarbonate, polystyrene, polypropylene, other polymer, etc). A secondary container may also be manufactured from the same material and/or as a part of the primary container. The pressure at which the secondary container will be disrupted will be defined by the rigidity of the secondary container material and the amount of sample and other compressible material (e.g., gas (e.g., inert gas e.g., helium, argon, neon, etc.), air, nitrogen, carbon dioxide, oxygen) inside the secondary container. The secondary container will release its contents (e.g., rupture) at the pressure levels at which its resistance to compression will be lower then the compressibility of its contents. The secondary container may also be made, e.g., out of an amorphous or crystalline compound, the melting point of which is above the sample processing temperature at atmospheric pressure. Application of high pressure will melt the secondary container material. Alternatively, the entire secondary container may be prepared out of the ingredient to be added to the mixture of liquid reagents, e.g., solid ice, solid lipid, paraffin, etc. Such material will become liquid under pressure and can participate in the extraction. It may or may not solidify again upon de-pressurization of the mixture. If this component does solidify and if it will contain several constituents of the initial mixture, which partitioned into it under pressure, the components can be fractionated out of the mixture by simple removal of the solidified material out of the mixture. The secondary container can be a nanomaterial (e.g., e.g., a suspension of micro-encapsulated reagent, reagent absorbed by porous materials).

Sources of Components for Extraction (Samples)

The extraction methods described herein can be used to extract a component of interest (e.g., molecular complex, e.g., organelle) from a sample.

Examples of sources from which a component can be extracted include biological and synthetic (e.g., man made, artificial cell, liposome) sources. Examples of sources of biological origin include animal (e.g., human, wild or domesticated animal, fish, bird, reptile, amphibian, insect, arachnid, mollusk, etc.), fungal, bacterial, viral, and plant sources. Examples of such sources include a cell, a membrane (e.g., a lipid membrane, e.g., a lipid bilayer), a biological sample (tissue sample, e.g., adipose tissue, liver, kidney, skin, pancreas, stomach, intestine, colon, breast, ovary, oocyte, testis, uterine, prostate, bone, feather, tendon, cartilage, hair, nail, scale, tooth, heart, brain, lung, gill, skin, nerve, biopsy, etc., blood, urine, milk, semen, saliva, venom, mucus, other bodily secretions, fluids and solids), samples that have been pre-processed prior to extraction, e.g. cooked food, or enzyme-digested tissues or a collection of cells (e.g., blood, semen, mucus, saliva, or tissue biopsy).

Pressure cycling extraction is suitable for use on liquid or solid tissue (soft or hard) samples. In some embodiments, soft tissue is preferred over hard tissue. For example, liver, kidney, ovary, pancreas, and brain may be easier to process than harder tissues such as muscle, intestine, heart, adipose, skin, hair, finger nail, bone and cartilage tissues. Cultured cells and body fluids, including blood, serum, urine and spinal fluid, can be processed as well.

Extracted Components

Examples of components (e.g., molecular complexes) that can be extracted by the methods described herein include organelles (e.g., mitochondrion, nucleus, Golgi apparatus, chloroplast, endoplasmic reticulum, vacuole, acrosome, centriole, cilium, glyoxysome, hydrogenosome, lysosome, melanosome, mitosome, myofibril, nucleolus, parenthesome, peroxisome, ribosome, proteosome, microsome, vesicle), a protein complex (e.g., that contains two or more proteins), a membrane channel, a membrane pore, a transcription factor complex, a cytoskeletal structure, a signal transduction complex, and a sub-organelle structure, such as mitochondrial inner membrane and its contents.

The characteristics of molecular complexes are the involvement of two or more molecules and/or two or more types of molecules in each complex. The molecular complexes can include biomolecules, such as protein-protein, protein-lipid, protein-nucleic acid, protein-peptide, protein-small molecules, nucleic acid-small molecules, and lipid-protein complexes. Molecular complexes other than commonly named “organelles” include free and bound complexes inside and outside of cells and tissues, associated with or separated from organelles. These complexes may be held together in kinetically rapid or slow fashions. The studies and applications of molecular complexes include the elucidation of biological networks under physiological and pathological conditions, drug discoveries, and clinical diagnostics.

By applying optimized pressure cycling conditions, one may extract not only a certain type of molecular complex or organelle, but also may enrich for a subset of a certain type of complex. For example, cells may contain subsets of different mitochondria, which have different structures and/or biochemical compositions. Subsets of mitochondria may have different thermal and/or pressure stability. Therefore, pressure mediated extraction may allow some subsets of mitochondria to be disrupted, while maintaining other subsets of mitochondria intact. This may be useful in elucidating and/or studying biological properties of various populations of mitochondria in a sample. The same principle can also apply to extracting molecular complexes and subgroups of cells.

Additional examples of extractable components are as follows:

TABLE 1
Prokaryotic organelles and cell components

| Organelle/Macromolecule | Main function | Structure | Organisms |
|---|---|---|---|
| carboxysome | carbon fixation | protein-shell | some bacteria |
| | | compartment | |
| chlorosome | photosynthesis | light harvesting | green sulfur |
| | | complex | bacteria |
| flagellum | movement in | protein filament | some prokaryotes |
| | external medium | | and eukaryotes |
| magnetosome | magnetic orientation | inorganic crystal, | magnetotactic |
| | | lipid membrane | bacteria |
| nucleoid | DNA maintenance & | DNA-protein | prokaryotes |
| | transcription to RNA | | |
| plasmid | DNA exchange | circular DNA | some bacteria |
| ribosome | translation of RNA | RNA-protein | eukaryotes & |
| | into proteins | | prokaryotes |
| thylakoid | photosynthesis | photosystem | mostly |
| | | proteins and | cyanobacteria |
| | | pigments | |

TABLE 2
Major eukaryotic organelles

| Organelle | Main function | Structure | Organisms | Notes |
|---|---|---|---|---|
| chloroplast | photosynthesis | double-membrane | plants, | has some |
| (plastid) | | compartment | protists | genes |
| endoplasmic | modification and folding | single-membrane | all | |
| reticulum | of new proteins and | compartment | eukaryotes | |
| | lipids | | | |
| Golgi apparatus | sorting and modification | single-membrane | all | |
| | of proteins | compartment | eukaryotes | |
| mitochondrion | energy production | double-membrane | most | has some |
| | | compartment | eukaryotes | genes |
| vacuole | storage & homeostasis | single-membrane | eukaryotes | |
| | | compartment | | |
| nucleus | DNA maintenance & | double-membrane | all | has bulk of |
| | transcription to RNA | compartment | eukaryotes | genome |

TABLE 3
Other eukaryotic organelles and cell components

| Organelle/Macromolecule | Main function | Structure | Organisms |
|---|---|---|---|
| acrosome | helps spermatozoa | single-membrane | many animals |
| | fuse with ovum | compartment | |
| centriole | anchor for | Microtubule | animals |
| | cytoskeleton | protein | |
| cilium | movement in or of | Microtubule | animals, protists, |
| | external medium | protein | few plants |
| glyoxysome | conversion of fat into | single-membrane | plants |
| | sugars | compartment | |
| hydrogenosome | energy & hydrogen | double-membrane | a few unicellular |
| | production | compartment | eukaryotes |
| lysosome | breakdown of large | single-membrane | most eukaryotes |
| | molecules | compartment | |
| melanosome | pigment storage | single-membrane | animals |
| | | compartment | |
| mitosome | not characterized | double-membrane | a few unicellular |
| | | compartment | eukaryotes |
| myofibril | muscular contraction | bundled filaments | animals |
| nucleolus | ribosome production | protein-DNA-RNA | most eukaryotes |
| parenthesome | not characterized | not characterized | fungi |
| peroxisome | oxidation of protein | single-membrane | all eukaryotes |
| | | compartment | |
| ribosome | translation of RNA | RNA-protein | eukaryotes & |
| | into proteins | | prokaryotes |
| vesicle | miscellaneous | single-membrane | all eukaryotes |
| | | compartment | |

Extraction of Transient Molecular Complexes

Transient Molecular Complexes are non-covalent molecular complexes that are dynamically created and destroyed within the cells. Current level of understanding of cellular biology and the complex interactions is primarily based upon easily characterized static models and typically does not take into account kinetics of formation and disassembly of such transient complexes. Understanding interactions between components within transient complexes is essential for robust modeling that can accurately describe development and progression of many.

It is desirable to monitor the dynamics of molecular complex formation and decay in real-time. For example, in vitro images of the formation of molecular complexes are captured using ultra-fast imaging systems. The disadvantage of this approach is that the information captured may not correspond to the actual reactions in vivo. Molecular complexes formed in cultured cells may be studied based on in situ fluorescence resonance energy transfer (FRET) imaging method. However, one of the technical difficulties is the introduction of FRET molecular probes, as cellular metabolism may be influenced by the presence of the FRET reagents. Because of the resolution limitations, it is also difficult to monitor the concurrent dynamics of the multiple molecular complexes.

Molecular biology techniques are frequently used in the studies of molecular interactions. For example, in the studies of signal transduction pathways, methods commonly include in vivo methods based on either removal (knockout) or introduction of the additional (transfection) potential partner proteins. Biological responses as a result of such intervention are measured. In some cases, labeled proteins or substrates are employed in measuring the dynamics of protein complexes and the affinities of their respective component molecules. Immunoprecipitation approaches have also been developed for isolation of interacting molecular complexes. Additionally, in vitro fluorescence resonance energy transfer (FRET) methods are employed for real-time measurements of complex formation and destruction. For in vitro studies, protein partners or effectors are identified by observing interactions with known isolated target proteins, e.g., receptors. The most popular pull down systems include the yeast two hybrid screening, phage display library screening, and GST systems. Surface plasmon resonance is also used in determination of binding kinetics and affinity of molecular complexes. Complementary data obtained via both in vivo and in vitro studies are typically needed in order to provide sufficient evidence and be confident in data interpretations. Alternatively, biologically-significant molecular complexes could be analyzed as isolated from metabolically quenched, or shut-down biological samples. Such an approach can be useful in identification and quantification using mass spectrometry (MS) or immunochemical techniques.

The pressure cycling methods of extraction, as described herein, can be used for isolation of transient molecular complexes. First, this method allows complexes to be extracted and quenched, fixed or chemically cross-linked concurrently with the extraction. For example, a sample is lysed and extracted with pressure cycles, in the presence of a secondary container which holds a protein fixation reagent, e.g. formalin, paraformaldehyde, acetone, or ethanol. The crucial element of such a procedure is that the molecular complexes are diluted enough in an extraction solution to permit limited fixation. If the solution is too concentrated, the cross-linking reaction may react non-specifically with random protein molecules, making identification of specific partners of a complex more difficult.

Thermodynamically speaking, the formation of a complex or interaction at high hydrostatic pressure is more favorable, when a complex occupies a smaller volume than the individual interacting molecules. Thus, the pressure cycling extraction is more suitable for the isolation of such complexes as they are more likely to be preserved during pressurization. However, complexes that occupy greater volume than their corresponding interacting parts are thermodynamically less favorable at high pressure. These complexes would be quickly deformed or dissociated. Preservation of these types of complexes would require modifications of the pressure cycling protocol. For example, by reducing the temperature to 4, −10 or −30° C., the dissociation of a complex may be slowed down. Protein stabilization agents may also be introduced, e.g., glycerol or ethylene glycol. The key is to stabilize the complex and let the fixation occur to a limited extent. Therefore, advantages of the pressure cycling method are related to its ability to differentiate thermodynamically between molecular complexes depending on the complex type, the affinity of interacting components (e.g., molecules) to each other, and the nature of the molecular interactions involved in a formation of said complex. Such differentiation could be optimized and precisely controlled by thermodynamic parameters of the process (temperature, pressure, and/or time), chemical composition of a surrounding media (e.g., buffer pH, ionic strength, concentration of detergents, organic solvents, chaotropic reagents, etc.) to reproducibly protect, extract, and isolate certain transient molecular complexes.

Sample collection methods for studies of transient molecular complexes described above can be very important, because during the collection, the metabolic state of the sample origin (e.g., organism, tissue, cell), composition, and quantity of molecular complexes of interest can be drastically affected. The samples collected for the study meet one or more of the following criteria. First, the sample to be examined must be preserved (e.g., rapidly preserved) (e.g., frozen, metabolically quenched or shut-down) so that there is a high probability of capturing molecular complexes formed during, or present at, a specific metabolic state. Second, samples appropriate for the transient molecular complex studies may be in various forms, e.g., fresh frozen tissues or cells, freshly preserved organs, their fragments, pieces of tissue, or fresh cultured cells. When molecular complexes are extracted, the sample is supplemented with a lysis buffer and possibly a pressure-responsive secondary container that holds a secondary reagent or multiplicity of reagents. The reagent from the secondary container is mixed with the primary lysis solution by pressure pulses and/or additional physical mechanisms, such as turbulent flow introduced by physical barriers, agitation by vibration, beads, magnetic stir bars, moving components in the sample container, etc. The secondary container may also be in the form of various nanomaterials (e.g., a suspension of micro-encapsulated reagent, reagent absorbed by porous materials) present in the lysis chamber with the sample at the time of experiment. Such nanomaterials (e.g., secondary containers) may be engineered to release reagents at specific pressure levels.

In clinical diagnostic applications, samples are often biological fluids, or biopsy tissues, e.g., tissues obtained by needles, laparoscopic or endoscopic equipment, or post-surgical tissue/organ specimens. Pressure cycling extraction could be beneficial for these applications. The advantages of this method include prompt sample preservation and processing; post-extraction preservation of targets; quantitative recovery of targets; broad compatibilities; and tunable processing parameters for selective preservation of certain targets (e.g., molecular complexes of interest) concurrent with selective dissociation of other molecular complexes (e.g., complexes that are not of interest). In some cases, extracted samples may be analyzed by HPLC, capillary electrophoresis, gel electrophoresis or other separation techniques. The comparative chromatogram profiles of the separation may be used in diagnostic studies.

Additional Processes Before and/or after Pressure Extraction—Pre-Treatment and Fractionation/Purification

A molecular complex that is extracted by a method described herein can be cleaned, polished, fragmented, or further fractionated or purified, before or after performing a method described herein. For example, a washing, grinding, mincing, culturing, incubation for temperature and/or solvent-exchange equilibration, centrifugation, and/or filtration step can be performed prior to the pressure cycling extraction. Likewise, a number of methodologies can be applied for fractionating molecular complexes after the pressure process, e.g., centrifugation, chromatography including buoyant density accumulation (BDA), sucrose gradient separation, HPLC, affinity binding chromatography, SEC, electrophoresis, filtration, washing, incubation at a temperature and/or solvent-exchange equilibration, enzymatic treatment and dialysis. In addition to theses methods, a molecular complex that is extracted by a method described herein can be further fractionated by a second round of pressure cycling using the same or different pressure conditions and chemistry.

For example, pressure-extracted mitochondria, extracts of heart tissue, lymphoblasts, yeast, or bacteria can be subjected to SDS-PAGE analysis. From mitochondria, all the multi-protein complexes of the oxidative phosphorylation system can then be separated using one gel. The complexes can be resolved into the individual polypeptides by second-dimension SDS-PAGE for component analysis, or fragmented with a native PAGE in the absence of SDS for studies of molecular complex activity or function. The percentage of the recovered functional activity may be estimated based on the respective protein complex of interest and some other complexes as internal control standards that may be quantitatively determined following the fractionation and purification procedure. The quantitative recovery of molecular complexes and immediate functional assay may be an especially useful approach for multiple purposes, e.g., separation of radioactively labeled membrane proteins, N-terminal protein sequencing, preparation of proteins for immunization, or diagnostic studies (e.g., of inborn neuromuscular diseases).

Alternatively, pressure-released mitochondria from cells or tissue may be suspended in a lysis buffer or other reagent and subjected to second round of pressure cycling in order to extract and/or purify mitochondrial components. Another example of pressure cycling followed by a second round of pressure cycling is the depletion of blood-derived proteins from tissue by pressure cycling in mild or physiological buffer, followed by extraction of tissue proteins by pressure cycling in a detergent-containing or other lysis buffer.

Analysis and Detection of Extracted Components

The extracted component (e.g., molecular complex of interest) can be analyzed for various purposes using methods known in the art. For example, methods for the analysis of molecular contents (e.g., of the molecular complex) include one-dimensional gel electrophoresis, two-dimensional gel electrophoresis, Western blotting and other immunological methods such as ChIP and ELISA, protein or peptide mass fingerprinting (e.g., using MALDI-TOF/TOF), multi-dimensional electrophoresis (e.g., solution phase isoelectric focusing followed by two-dimensional gel electrophoresis of concentrated pI fractions), mass spectrometry (MALDI-MS, LC-MS/MS, MALDI-TOF MS, or LC-ESI-MS/MS), PCR (e.g., real-time PCR), RT-PCR, microarrays, thin-layer chromatography, liquid chromatography, gas chromatography, GC/MS, electron microscopy, fluorescent microscopy, and surface analysis methods. In certain embodiments, isolated molecules or complexes thereof or organelles may be used in functional assays, e.g., enzymatic activity assays, ion channel function assays, molecular gating assays, in vitro metabolism assays, cytotoxicity assays, etc., or subjected to subsequent fractionation or extraction steps. Further, genomic or proteomic analysis can be performed on nucleic acids or proteins present in the extracted molecular complex (e.g., mitochondrial DNA or proteins from extracted mitochondria can be analyzed).

Examples of downstream processing include: evaluation of a molecular complex for the presence or absence of a component (e.g., a protein, an enzyme, a DNA sequence (e.g., a mutation, methylation, and other adduct), an RNA sequence (e.g., a mutation, maturation), or a metabolite; analytical discoveries, diagnostics, preparation of products, drug discoveries, organelle biochemical function and activity, and pharmacokinetics at the molecular complex levels. In addition to component analysis of molecular complexes, an important application of the pressure extraction methods relates to functional studies and applied assays in biological, biomedical and pharmaceutical areas. For example, organelles isolated from fresh animal tissues using this pressure extraction method can be used as standard material in organelle functional assays.

Additional Uses of Pressure Cycling Technology in Molecular Complex Extraction

Examples of additional uses for the methods described herein include

Pressure Cycling in Conjunction with Osmotic Pressure

The isolation of organelles, such as nuclei, from cells is often performed by placing the cells into a hypotonic buffer, causing the cells to swell and become more fragile. Cell lysis is then carried out by mechanical means such as homogenization. This method, while effective, is highly variable and can be damaging to the target being isolated (e.g., nuclei or other sub-cellular target). By placing the sample (e.g., mammalian cells and tissues) into a hypotonic solution (water or other appropriate buffer), and performing pressure cycling in that solution, it can be possible to more efficiently and reproducibly isolate the target or targets, because swollen cells are more prone to lysis with pressure cycling. In this way, it may be possible to use relatively mild pressure cycling conditions, which will allow better recovery of fragile targets.

Hypertonic buffers may be more beneficial for cells surrounded by a rigid cell wall (e.g., plant cells, bacteria, fungi) as hypotonic swelling normally leads to an elevated hydrostatic pressure inside each cell (turgor) and therefore antagonizes pressure cycling. Conversely, shrinking of the cell in a hypertonic solution may provide better starting conditions for pressure cycling.

Separation of Infecting Micro-Organisms from Infected Cells and/or Tissue

Because certain cell types, such as eukaryotic cells, are frequently more susceptible to lysis under pressure than certain other cell types, such as some micro-organisms (e.g., parasites, bacteria, or viruses), it is possible to isolate the infecting agent intact from the infected cells or tissue, by pressure cycling. That is, if a population of infected cells is treated with pressure cycling under conditions that are sufficient to lyse the infected cells or tissue and release the infecting agent, but mild enough so as not to destroy the agent, then the agent can be isolated intact. These released microorganisms may be collected and further enriched by sedimentation, or immuno-precipitation, or cultured, or detected by staining, nucleic acid amplification and detection techniques (e.g. PCR, TMA, rt-PCR, etc.). Since only large complexes, such as the intact microorganisms or certain organelles may be easily isolated by centrifugation or filtration, the collected sample may be suitable for direct PCR amplification and sequence analysis without isolations of the purified nucleic acids.

Differential Lysis of Cells Based on Viral Infection Status

Because virus-infected cells frequently have altered plasma membrane properties as a result of viral infection (e.g., due to virus shedding at the plasma membrane, overexpression of viral proteins or other effects of infection), pressure cycling of a mixed cell population containing both infected and uninfected cells under certain conditions (which may vary for each specific cell and virus type) can allow the selective lysis of one cell type (e.g., the infected cells), while the other cell type (e.g., the uninfected cells) remains intact. Such an application can be used both in clinical or research laboratory environment. For example, the procedure could be used for clinical applications, e.g., to selectively deplete virus-infected cells from blood, bone marrow or other organs prior to transplantation (autologous or heterologous). Non-clinical applications could include research, e.g., by depleting infected cells in a mixed population of infected and non-infected cells, the enriched population of non-infected cells can be used to study the molecules or mechanisms that make those cells more resistant to infection by the virus in question.

Cell Culture Synchronization by Pressure Cycling

Because the sensitivity of cells to lysis induced by pressure cycling can vary depending on the cell cycle status of the cells, it may be possible to use pressure cycling to synchronize mixed populations of cells by selectively killing or inactivating cells at certain phase(s) of the cell cycle (e.g., growth phase, mitosis, meiosis, senescence, etc.), while leaving intact cells at a different phase. In this way, the entire surviving viable population of treated cells can be synchronized to the same phase of the cell cycle.

Differential Lysis of Cells by Pressure Cycling Based on Cell Differentiation

Because the sensitivity of cells to lysis induced by pressure cycling can vary depending on the cell cycle and/or differentiation status of the cells, it may be possible to use pressure cycling to separate cells based on whether they are more differentiated (e.g., normal somatic cells, differentiated cell culture cells or tissues), or less differentiated (e.g., some tumors or tumor cells). For example, if a population of cells containing both normal cells and tumor cells is treated with pressure cycling under conditions that are sufficient to kill or inactivate one cell type (e.g., the tumor cells) but mild enough to preserve the other cell type intact (e.g., the normal cells), then a population of cells can be isolated that is enriched for (or is purely composed of) one of the cell types in the original mixed population (the desired cell type), and depleted in the other cell type (the contaminating cell type). This can be useful in both clinical and research applications.

Enrichment for Stem Cells from a Population of Mixed Cells or Tissues

Because the sensitivity of cells to lysis induced by pressure cycling can vary depending on the cell cycle and/or differentiation status of the cells, it should be possible to use pressure cycling to enrich for less differentiated or undifferentiated cells (e.g., adult stem cells or embryonic stem cells) in a mixed population containing differentiated cells (e.g., normal somatic cells, differentiated cell culture cells, or tissues). For example, if a population of cells containing both stem cells and more differentiated cells is treated with pressure 5 cycling under conditions that are sufficient to lyse the differentiated cells but mild enough to preserve the viability of the stem cells, the stem cells can be isolated intact.

Enrichment for Viable or Intact Bacterial or Viral Material from Cells or Tissues

This method is based on the higher sensitivity of animal cells than bacterial or viral cells to pressure cycling. Such sensitivity relates to both cell death and disruption, which for animal cells may happen at distinctly different levels of pressure than inactivation or disruption of bacterial or viral infective agents. Resulting cell debris and bacteria/virus can be separated by differential centrifugation.

### EXAMPLES

### Example 1

### Cell Lysis: Comparison of Continuous Versus Cycled Pressure

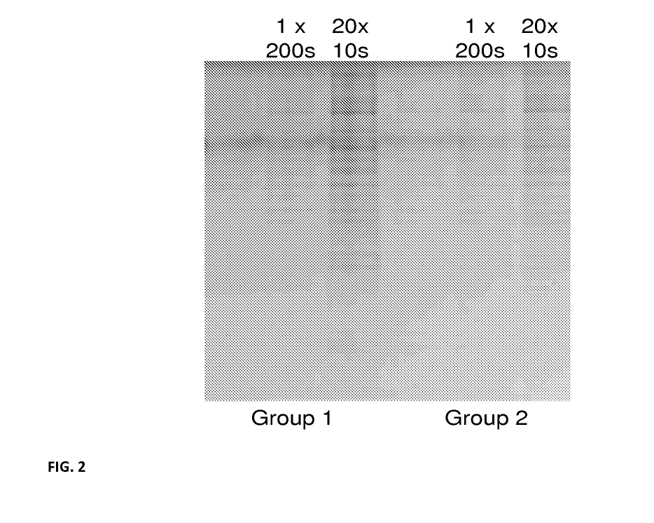

In order to demonstrate that cellular contents can be efficiently released by Pressure Cycling Technology (PCT), cells were treated with either a single long pulse, or multiple short cycles, of high pressure. PC12 cells were pelleted, washed and resuspended in PBS. The cell suspensions were aliquoted into PULSE™ tubes and pressurized to 25 kpsi either once for 200 seconds, or by pulsing 20 times for 10 seconds at 25 kpsi, with 10 seconds at atmospheric pressure between each pulse. In both cases the cells were exposed to a pressure of 25 kpsi for a total of 200 seconds. After treatment, aliquots of each sample were removed for cell counting, and the remaining suspension was centrifuged to separate the intact cells from the supernatant containing soluble cytosolic proteins released from lysed cells. This supernatant was loaded onto SDS-PAGE and the resulting gel was stained with Coomassie Blue to visualize total released protein from lysed cells (FIG. 2). Results show that both the cell counts (data not shown) and the SDS-PAGE results agree and indicate that cell lysis is induced much more efficiently by cycles of high and low pressure (<66% intact cells vs. untreated control), than by a single continuous exposure to high pressure (>90% intact cells vs. untreated control).

### Example 2

### Cycle Profile in the Recovery of Mitochondria Monitored Using q-PCR

200 mg of liver tissue was processed in 1.1 ml HEPES buffer (20 mM HEPES, 5 mM MgCl2, 250 mM Sucrose, 1 mM EDTA, pH 7.3) using the Pressure Cycling Technology (PCT) Sample Preparation System (BAROCYCLER™ NEP3229 and PULSE™ Tubes, FT500). Pressure cycling was performed at various pressure levels, ranging from 5 to 35 kpsi, and number of cycles, ranging from 0 to 30, at 4° C. Pressure incubation periods were 20 sec at high pressure and 10 sec at atmospheric pressure. The pressure extraction was compared with processing in a Dounce homogenizer. Following the extractions, samples were transferred to microcentrifuge tubes and centrifuged at 800×g, 4° C. for 10 minutes (1^{st} and low speed spin). Then supernatants were collected and centrifuged at 8,000×g for 10 minutes (2^{nd} and high speed spin). The pellets were harvested and washed with HEPES buffer by centrifugation at 8,000×g for 10 minutes (3 and post-washing spin). The mitochondria-containing pellets were then purified for mitochondrial DNA (mDNA) using Qiagen DNeasy kit. The mitochondrial DNA of each sample were eluted with 200 μl DEPC-treated ddH₂O 1.0 μl of DNA from each sample was used in a quantitative PCR (Q-PCR) assay using primers and probe detecting the mitochondria 12S rRNA gene. The copy numbers of mDNA were determined based on standard curves from known concentration of templates. The results are shown in FIG. 3.

As shown in FIG. 3, higher yields of mitochondrial DNA were recovered after PCT at lower pressure, (i.e., 5 or 10 kpsi) and larger number of cycles (i.e. 10 or 20 cycles). When PCT was increased to 30 cycles, the mDNA yields decreased again and approached the level of those recovered using 5 cycles. This suggests that there is an increase in the number of damaged mitochondria.

There are at least two factors operating at the tested pressures and cycle numbers. There is a competition between more efficient tissue disruption and accumulating mitochondrial damage. In the case of frozen-thawed rat liver, the 5 kpsi, 5 cycle process may be more efficient for recovering intact mitochondria. For other applications, PCT conditions can be optimized/adjusted by the user.

### Example 3

### Pressure Profile of Mitochondria Extraction

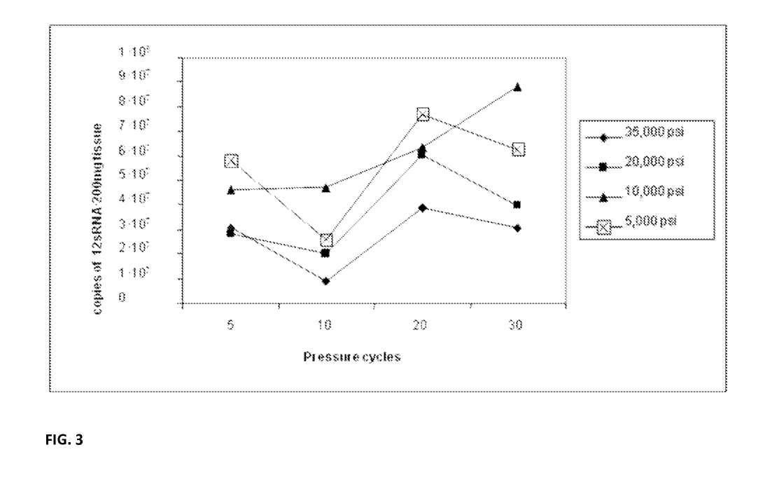

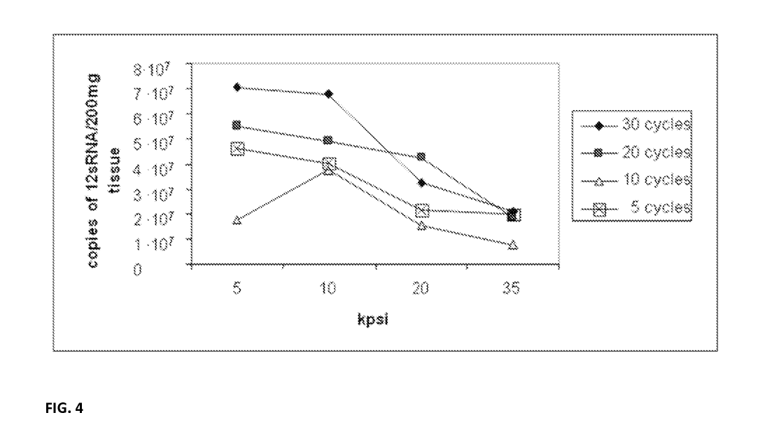

These samples were processed similar to those shown in FIG. 3. Here, a constant number of cycles were used. As shown in the previous studies, the mDNA yields increased using a larger number of pressure cycles. However, more damage to the mitochondria may also occur using a larger number of cycling. This was suggested based on a morphological examination by Transmission Electron Microscope (TEM) (Example 13). FIG. 4 shows that less mDNA was recovered at higher pressure with the same number of cycles. This result suggests that higher levels of pressure cause pressure-related mitochondrial damage and lead to decreased yield.

### Example 4

### Mitochondria Recovery at Various Centrifugation Speeds

After extraction, centrifugation can be used to obtain ‘semi-purified’ organelles. In this example, the correlation of the centrifugation speed and the yield of mitochondrial DNA recovery from the pressure cycle produced extracts were analyzed.

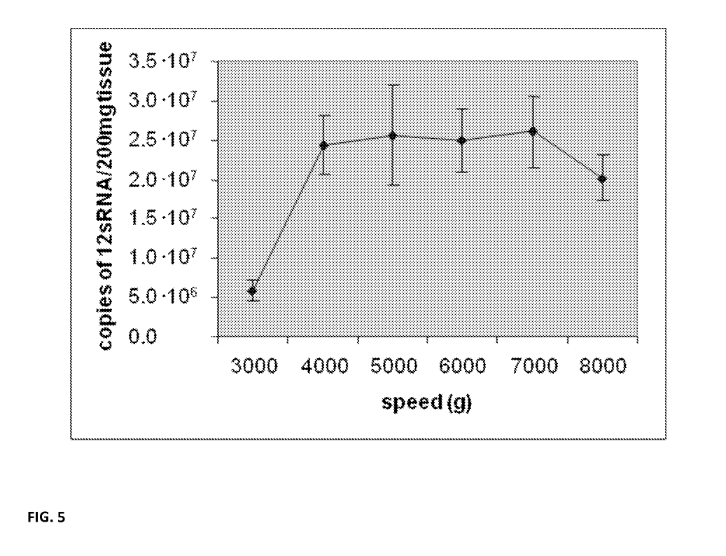

The final mitochondrial separation was carried out using two centrifugation steps. First, the sample was centrifuged at low speed (800×g), 4° C. to spin down the cell debris. At the second step, the supernatant obtained from the first step was centrifuged at a higher speed (3,000-8,000×g), 4° C. The results are shown in FIG. 5.

The results show that at 3,000×g, the mDNA yield was lower. Between 4,000 to 8,000×g, the mDNA yields were increased and similar levels seemed to be recovered at all speeds. In terms of mitochondria yields, speeds above 4,000×g appeared to result in the same yield of mitochondria.

### Example 5

### Different Sample Input Amounts and mDNA Recovery

The goal of this experiment was to determine whether mDNA recovery correlated with input amount of tissue.

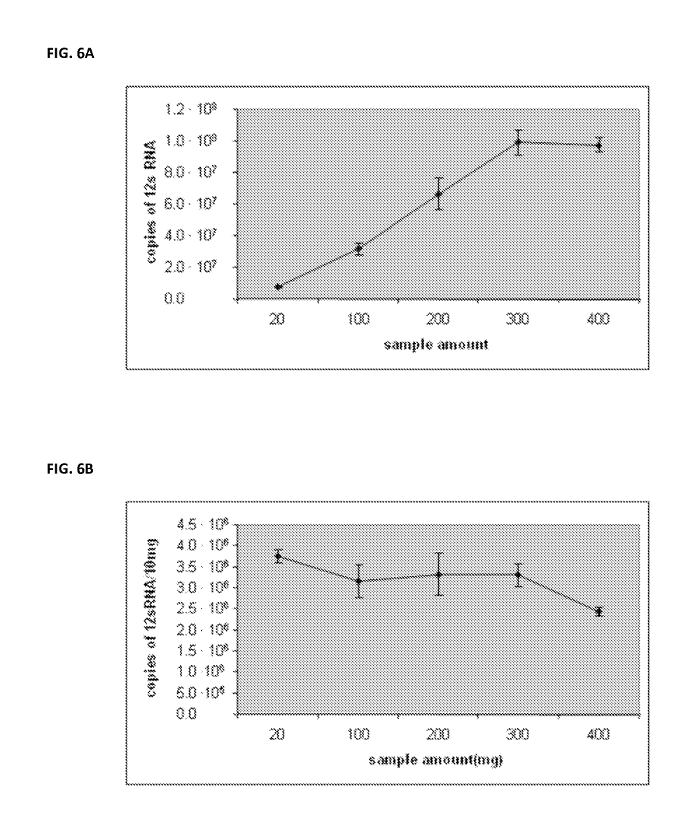

Different amounts of rat liver were processed in FT500 PULSE™ Tubes (Pressure Biosciences, Inc.) by pressure cycling between atmospheric and 5 kpsi of pressure, 5 cycles at 4° C. Incubation periods were 20 sec at high pressure and 10 sec at atmospheric pressure. After removing insoluble material and larger organelles by centrifugation at 800×g, 4° C., mitochondria were harvested by a 2^{nd} centrifugation at 5,000×g for 10 minutes, 4° C. The final pellet was resuspended in 400 μl PBS buffer. 200 μl mitochondria suspension was used to purify mitochondria DNA. The final DNA was eluted in 200 μl ddH₂O. 1.0 μl mDNA was used for Q-PCR assay. The results are shown in FIGS. 6A and 6B.

As shown in FIG. 6A, when liver samples were less than 300 mg, the mDNA yield was linearly proportional to the amount of starting material, which suggests that within a certain range, recovery increases linearly with sample mass, therefore PCT can be considered a quantitative extraction technique.

### Example 6

### Improving mDNA Recovery by Exchanging Fresh Extraction Buffer and Repeated Pressure Cycling Extraction

This experiment aimed to determine whether mDNA recovery can be improved by repeated rounds of pressure cycling.

Samples (200 mg of rat liver) were repeatedly processed under the condition of 5 kpsi and 5 cycles as described in the Example above. After each round of 5 cycles, the supernatant was collected in a separate container after centrifugation at 800×g in the PULSE™ Tube. Fresh buffer was added to the PULSE™ Tube and another round of 5 cycles was performed.

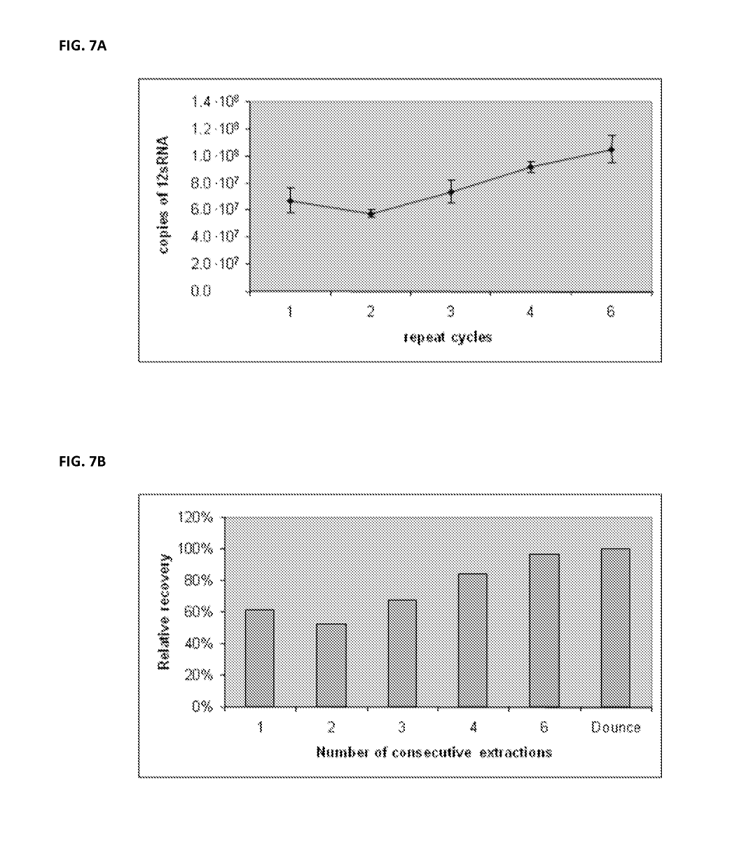

To increase mitochondria yields using pressure cycling, cycle number and pressure conditions in a single pressure cycling process were tested, as shown in FIG. 3 and FIG. 4. However, these results showed that by increasing pressure levels, mDNA yield was decreased. mDNA yield may be slightly increased by increasing cycle numbers at relatively low pressure. However, increasing pressure and the number of pressure cycles appears to cause increased mitochondria damage. This result may represent a balance between release from the cells and mitochondrial damage. Therefore, an alternative approach, using several rounds of pressure cycling was tested to increase mitochondria yield while minimizing damage. This was accomplished by carrying out several rounds of pressure cycling at one of the ‘mildest’ conditions, i.e., 5 kpsi and 5 cycles for each round as described above, harvesting the supernatant and adding fresh buffer for another processing. The results are shown in FIGS. 7A and 7B.

As shown in FIG. 7A, the total yields of mDNA using pressure cycling increased by repeating the pressure cycling process. The increase in yield is proportional to the repeat number. Compared to processing in a Dounce homogenizer, one round of pressure cycling process improved the yield of mitochondria by about 60% (FIG. 7B). The advantage of using pressure cycling may be the quality, reproducibility and intactness of mitochondria.

### Example 7

### Extraction Buffer Composition and mDNA Recovery

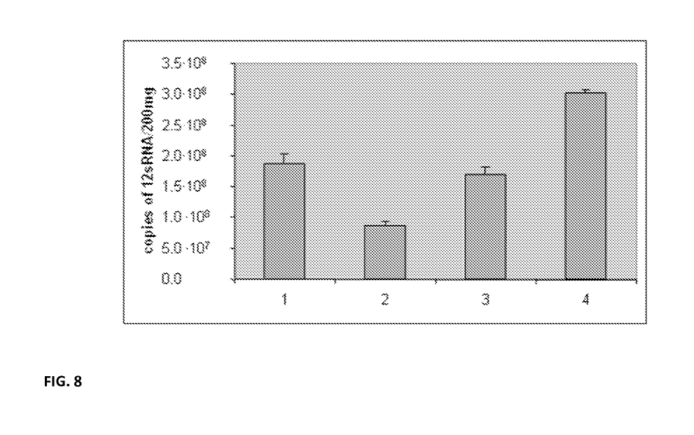

In this experiment, 200 mg of rat liver tissue with different buffers were processed using pressure cycling under the condition of 5 kpsi, 5 cycles as described above. 200 mg of liver tissue was also processed by the Dounce homogenization method. The results are shown in FIG. 8.

As shown in the figure, similar mDNA yields were obtained by using PBS and HEPES supplemented with 250 mM sucrose as the extraction buffer (compare Sample No. 1 and 3). However, with HEPES alone, the yield of mitochondria was only half as much as the other buffers. This suggests that PCT methods of mitochondrial extraction are compatible with a wide variety of buffer systems compatible with organelle isolation, but that it is important to maintain the osmotic balance during the mitochondria extraction.

### Example 8

### Mitochondria Isolation

When there are cellular structures that may protect cells from extraction, (e.g., collagen in a tissue, or cell walls of thick polysaccharides) hydrolytic enzymes may be employed to facilitate and improve the access of extraction buffer and air to the cells. For example, after several rinses with extraction buffer, e.g., PBS or HEPES, skeletal muscle can be suspended in 10 volumes (wt/vol) of the same buffer and treated with the protease nagarse (5 mg/g tissue) for 10 minutes on ice with constant stirring, and then treated with high pressure in the presence of the enzyme. After the pressure treatment at 5 cycles between atmospheric and 5 kpsi of pressure, the sample is diluted with an equal volume of extraction buffer supplemented with defatted bovine serum albumin (BSA) to 0.2% (wt/vol). Nagarse can then be removed by centrifugation (10 min. at an average of 7,802×g). The pellet is resuspended in PBS and subjected to a second pressure cycling process, e.g., 5 cycles between atmospheric and 5 kpsi of pressure as described above. Following the pressure treatment, the extract can be centrifuged at 800×g, 4° C. for 10 min to remove insoluble nuclei. The supernatant is centrifuged at 5,000×g for 10 min at 4° C. The mitochondria are subjected to two additional washes (5 ml BSA-supplemented isolation medium/g tissue and 2.5 ml of (in mM) 100 KCl, 50 MOPS, and 0.5 EGTA, pH 7.4/g muscle) and finally resuspended with 1.2 ml of (in mM) 100 KCl, 50 MOPS, and 0.5 EGTA, pH 7.4.

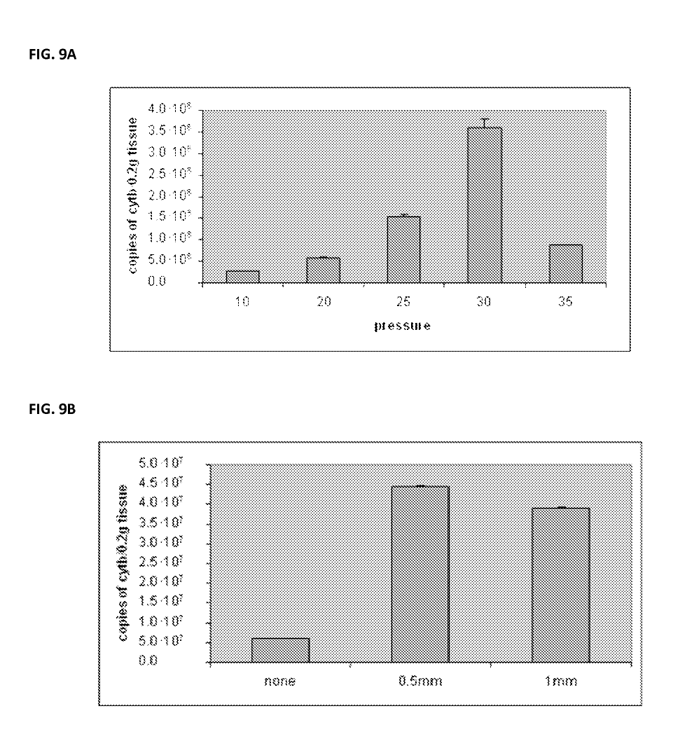

FIGS. 9A and 9B show the recovery of CytB mDNA from rat heart tissue. As compared to rat liver, heart is more difficult to extract, thus, higher pressure is necessary. However, pressure greater than 30 kpsi can actually lower the mitochondrial recovery, e.g., pressure at 35 kpsi (FIG. 9A). In another experiment, glass beads were also supplemented during the pressure extraction. It shows that the mitochondrial extraction was improved in the presence of 0.5 or 1.0 mm glass beads, comparing with no beads (FIG. 9B).

### Example 9

### Mitochondrial Proteome Analysis

Mitochondrial dysfunction, damage, and mutations of mitochondrial proteins give rise to a range of ill-understood patterns of disease. Although there is significant general knowledge of the proteins and the functional processes of the mitochondria, there is little knowledge of difference regarding how mitochondria respond and how they are regulated in different organs and tissues. Proteomic profiling of mitochondria and associated proteins involved in mitochondrial regulation and trafficking within cells and tissues has the potential to provide insights into mitochondrial dysfunction associated with many human diseases.

A proteomic study of mitochondrial proteome is as follows. 400 mg fresh-frozen rat liver samples were processed with 1.0 mL of HEPES buffer (20 mM HEPES, 5 mM MgCl2, 250 mM Sucrose, 1 mM EDTA, pH 7.3) with 1 pellet of protease inhibitor (Roche). These samples were treated with 30 cycles of pressure between ambient (10 sec) and 5 kpsi (20 sec) at 4° C. All lysates from these samples were collected, centrifuged at 1,000×g at 4° C. for 10 min. Supernatants were collected and loaded onto a BDA instrument (AWPT, W. Caldwell, N.J.). 96 BDA fractions were collected at the end of the BDA run. These fractions are analyzed using BCA protein analysis kit (Pierce Biotechnology, Rockford, Ill.), followed by Western blotting with MS604 antibody panel (MitoSciences, Eugene, Oreg.). Two pools of fractions were examined using 2DGE. The pressure cycling method was compared with the traditional Dounce homogenizer method. The results showed that pressure cycling extraction is a more reproducible method for recovering mitochondria than the Dounce homogenizer method. The Western blots showed good recoveries of five antigens probed using the MS604 panel, which indicates that intact mitochondria were recovered in the pressure cycling extracts.

### Example 10

### Mitochondrial Biological Function Assay

Cytochrome C oxidase is the principal terminal oxidase of high affinity oxygen in the aerobic metabolism of all animals, plants, yeasts, and some bacteria. The enzyme is present in mitochondria of the more highly developed cells and in the cytoplasmic membrane of bacteria. This enzyme is probably unique in providing energy for the cell by coupling electron transport through the cytochrome chain with the process of oxidative phosphorylation. Cytochrome C oxidase is located on the inner mitochondrial membrane that divides the mitochondrial matrix from the inter-membrane space and it has been used for many years as a marker for this membrane.

The calorimetric assay employed in this study is based on observation of the decrease in absorbance at 550 nm of ferrocytochrome c caused by its oxidation to ferricytochrome c by cytochrome c oxidase. A cytochrome C oxidase kit from Sigma (St Louis, Mo.) was purchased and used in this study. First, reduced ferricytochrome c is prepared by dissolving 2.7 mg of cytochrome c (MW 12,384 Da) in 1 ml of water. In order to reduce the protein, 5 μl of the 0.1 M DTT solution is added to a final concentration of 0.5 mM. The solution is mixed gently for 15 minutes. The color of the solution changes from dark orange-red to pale purple-red. The A550/A565 ratio is measured by diluting an aliquot of the solution 20-fold in a 1× Assay Buffer (e.g., 50 μl sample-to-be-tested in 950 μl of 1× Assay Buffer). The 1× Assay Buffer is used to zero the spectrophotometer. The A550/A565 ratio is typically between 10 and 20.

The effect of the number of pressure cycles at 5 kpsi on cytochrome C oxidase activity is shown in FIG. 10A.

For example, the effect of different pressures was tested on the cytochrome c activity of extracted mitochondria and mitochondria outer membrane integrity. The result demonstrated that mitochondrial cytochrome c activity decreased with increasing pressure between 5 and 35 kpsi (FIG. 10B). 200 mg rat liver tissue was processed under the pressures shown in the figure legend for 5 cycles under the condition of 20 seconds at high pressure and 10 seconds at atmospheric pressure, 4° C. At lower pressure, extracted mitochondria could maintain the highest cytochrome c activity.

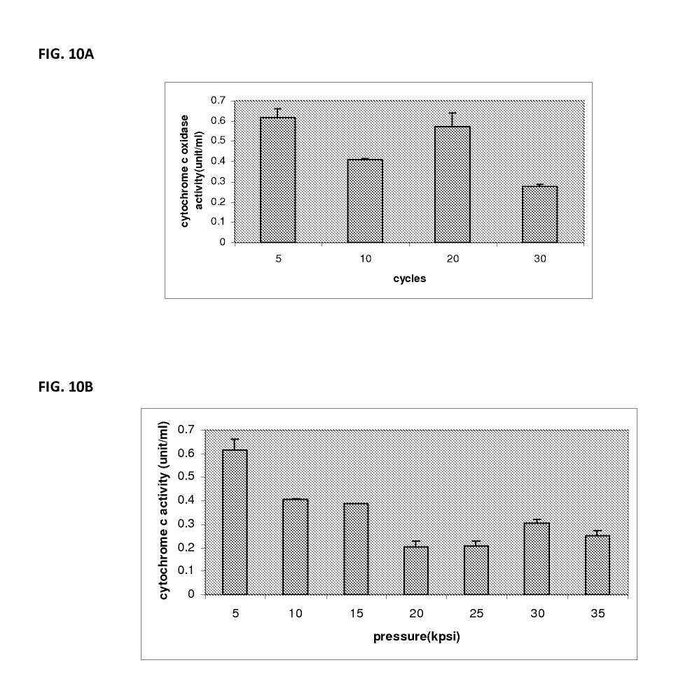

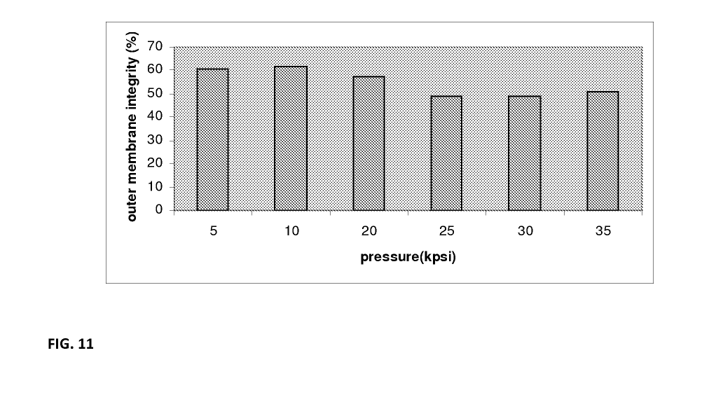

The mitochondria outer membrane integrity was also decreased with increased pressure (FIG. 11), but the decrease in membrane integrity was not closely correlated with the decrease of cytochrome c activity. 200 mg of rat liver tissue were processed under different pressures. Mitochondria outer membrane integrity was measured using a the same cytochrome c oxidase assay kit (Sigma). As shown in FIG. 11, the percent-outer membrane integrity was all between 50 and 60%. Although it decreased slightly, the observed outer membrane integrity remains similar at all the pressure levels tested. This finding indicated that, using higher pressure, the structure of mitochondria extracted with pressure cycling either were totally disrupted or kept intact, only a small fraction of the mitochondria were membrane damage, but still remained and collected after the second centrifugation at 5,000×g. This result also confirms that 5 kpsi and 5 cycles is a favorable condition for mitochondria extraction using pressure cycling.

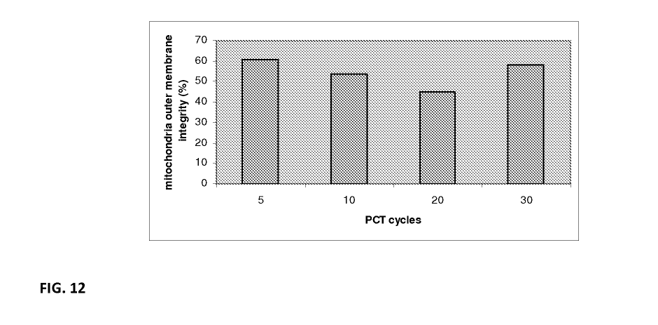

The influence of different pressure cycles was measured on the cytochrome c oxidase activity and outer membrane integrity. 200 mg rat liver tissue were processed using pressure cycling under 5 kpsi for different cycles. The results demonstrated that at 5 kpsi and 5 cycles, both cytochrome c oxidase activity and outer membrane integrity are at the highest level. Mitochondria outer membrane integrity was measured using a cytochrome c oxidase assay kit (Sigma). By increasing the number of pressure cycles, a decreased oxidase activity was observed. However, the outer membrane integrity of these samples was similar—between 50-60% (FIG. 12). This indicates that mitochondria may be disrupted at higher pressure; however, the remaining mitochondria after higher pressure extraction maintain minimal outer membrane disruption, which is an indication of the intactness of mitochondria. This result suggests that fewer cycles may be optimal for intact mitochondria extraction.

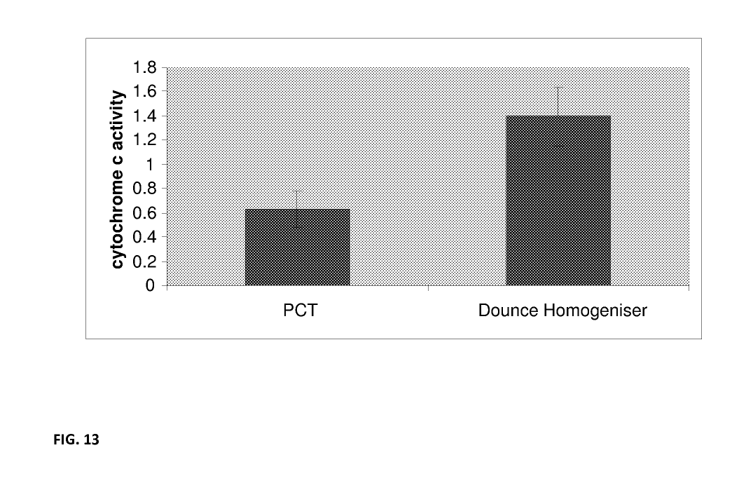

In another experiment, pressure cycling extraction of mitochondria was compared with Dounce homogenization. 200 mg liver tissue were processed by Dounce homogenizer (4 strokes) (n=4) or pressure cycling (5 kpsi, 5 cycles of 20 sec high and 10 sec low pressure) (n=10). The cytochrome c activity was measured using cytochrome c kit (Sigma). The results showed that cytochrome c oxidase activity using the Dounce method was higher than the activity after pressure cycling (FIG. 13). However, the mitochondria outer membrane integrity after using the Dounce method was poorer than that after pressure cycling. These results indicate that the Dounce method causes more mitochondria outer membrane damage than pressure cycling causes, despite a larger amount of mitochondria being obtained by the Dounce method. It was estimated that greater than 50% of the mitochondria extracted by Dounce homogenization suffered from outer membrane damage. In contrast, mitochondria extracted using pressure cycling maintained better mitochondria structure integrity.

### Example 11

### Cell Lysis by PCT: Recovery of Mitochondria from Lysed Cells

Mitochondria-enriched fractions were prepared from cells in culture. Cells were pelleted by centrifugation and brought up in either Mitochondrial Isolation Buffer 1 (MIB1) (10 mM sucrose, 200 mM mannitol, 5 mM HEPES, 1 mM EGTA, 1 mg/ml fatty acid-free bovine serum albumin), or Mitochondrial Isolation Buffer 2 (250 mM sucrose, 2 mM HEPES, 0.1 mM EGTA). Cells in each type of buffer were split into two PULSE™ tubes and processed by PCT using 2 sets of pressure profiles: 1). 30 sec at 25 kpsi followed by 20 sec at atmospheric pressure, repeated for 15 cycles; and 2). 30 sec at 15 kpsi followed by 20 sec at atmospheric pressure, repeated for 10 cycles. After cell lysis by pressure cycling, samples were centrifuged at low speed (900×g) to pellet any remaining intact cells and large cellular debris. The resulting pellet (pellet 1) was saved and the supernatant was transferred to a fresh tube and centrifuged at high speed (13,000×g) to bring down mitochondria and other small organelles (pellet 2). After the second spin, the mitochondria-enriched pellet and the supernatant, containing soluble cytosolic proteins, were saved. Aliquots of pellet 1, pellet 2 and supernatant from all four samples were separated by SDS-PAGE and either stained with Coomasie Blue dye for total protein visualization, or transferred to Immobilon-P for Western blotting. Duplicate Western blots were probed for either GAPDH, which is used as a loading control, or for VDAC/Porin, a component of the mitochondrial outer membrane (FIG. 14A).

Western blotting confirms that pellet 2 in all 4 samples is enriched for mitochondria. The difference in VDAC/Porin signal in either the 25 kpsi or 15 kpsi samples appears small. These results indicate that PCT is compatible with isolation of sub-cellular fractions enriched in certain components, such as mitochondria. These data also indicate that the PCT extraction method is compatible with different buffer systems and works efficiently in a range of pulsing conditions, which can be optimized to obtain sub-cellular fractions enriched in various cellular components.

In another experiment, mitochondria-enriched fractions were prepared from cells essentially as described above. Rat PC12 cell pellets were brought up in Mitochondrial Isolation Buffer 1 and split into four aliquots which were processed using 4 sets of conditions: 1). Atmospheric pressure control (“0” kpsi), 2) 30 sec at 5 kpsi followed by 20 sec at atmospheric pressure, repeated for 15 cycles; 3). 30 sec at 15 kpsi followed by 20 sec at atmospheric pressure, repeated for 15 cycles; 4). 30 sec at 25 kpsi followed by 20 sec at atmospheric pressure, repeated for 15 cycles. Pellet 1, pellet 2, supernatant and wash fractions were collected as described above and run on SDS-PAGE for Western blotting. To confirm that the mitochondria-enriched fractions contain intact mitochondria, blots were probed with 3 antibodies that recognize distinct mitochondrial compartments.

The results shown in FIG. 14B confirm that, in the absence of PCT (0 kpsi control fractions) all the mitochondrial signal is in the intact cells in pellet 1. Under PCT conditions the cells begin to lyse and, with increasing pressure, more and more of the mitochondria are released from the rupturing cells and can be recovered in pellet 2. The absence of a strong mitochondrial signal, especially the soluble HSP60, in the cytosolic supernatant, supports the conclusion that the PCT protocol is gentle enough to keep the bulk of the mitochondria intact, since large numbers of ruptured mitochondria would result in HSP60 leakage into the supernatant fraction.

The stringency of the pressure cycling conditions can be adjusted based on the downstream applications. PCT conditions can be adjusted to extract a smaller number of mitochondria under relatively gentle pressure, or a larger number of mitochondria using more intense pressure cycling.

### Example 12

### Isolation of Mitochondria from Fresh Rat Tissues by PCT

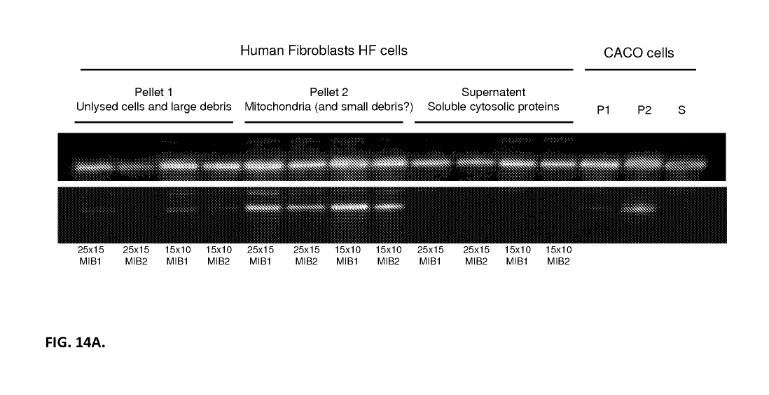

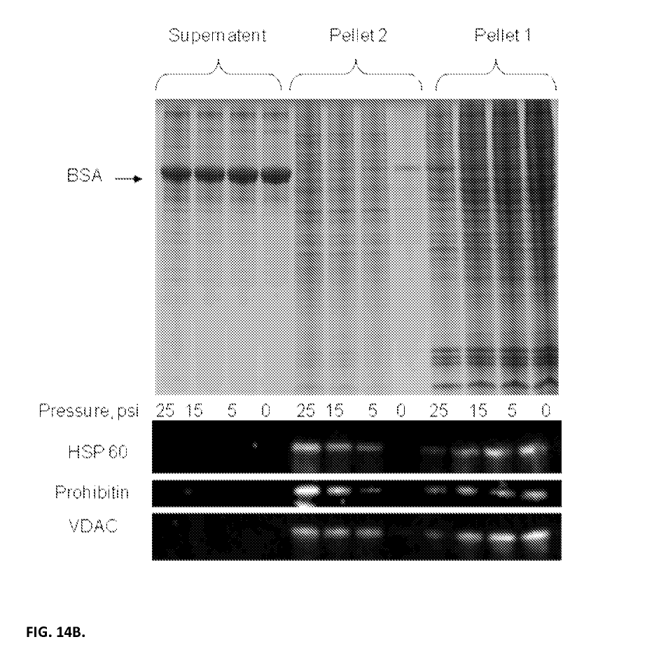

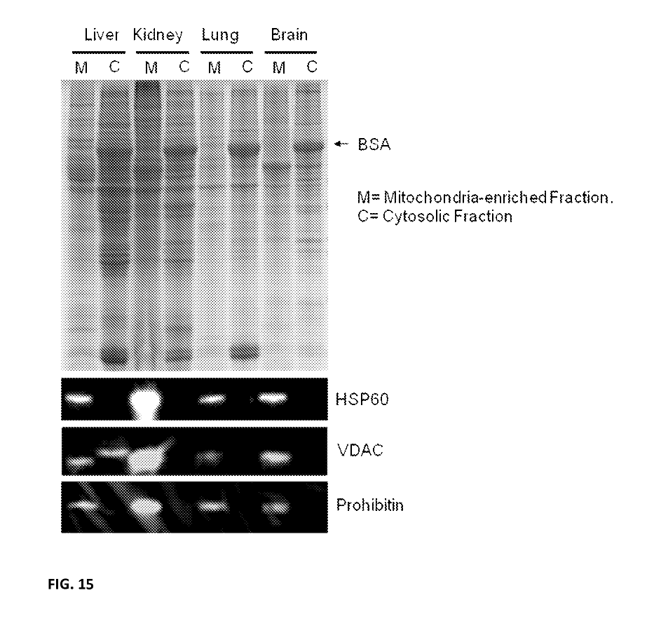

Freshly harvested rat tissues (liver, lung, kidney, and brain) as well as frozen mouse adipose tissue were processed for mitochondrial isolation by PCT. Tissues were rinsed in PBS to remove excess blood, and ˜200 mg of each tissue was used for processing. Tissues were placed into PULSE™ Tubes with a Mitochondrial Isolation Buffer (10 mM sucrose, 200 mM mannitol, 5 mM HEPES, 1 mg/ml fatty acid-free bovine serum albumin) and processed at 25 kpsi for 15 cycles (30 sec at high pressure, 20 sec at atmosphere). After PCT the lysates were centrifuged at low speed to pellet unlysed cells and debris. The supernatants were transferred to clean tubes and centrifuged at high speed to bring down mitochondria. The mitochondrial fractions (pellet 2) and the cytosolic supernatants from all tissues were loaded onto quadruplicate SDS-PAGE gels for Coomassie blue staining and Western blotting (FIG. 15)

Preliminary results using fresh rat tissues indicate that a protocol similar to that described above can be used to isolate a mitochondria-enriched fraction from various tissues including kidney, brain, lung, adipose and liver.

### Example 13

### Images of Pressure Cycling Extracted Mitochondria Using Transmission Electron Microscopy

Fresh mouse liver tissue was collected and stored on ice prior to pressure cycling extraction. 5 cycles of pressure between 5 kpsi and ambient at 4° C. were applied to 100 mg liver tissue in HEPES buffer (recipe shown above). After eliminating debris and nuclei by centrifuging the lysates at 800×g, 4° C., 10 min, the supernatant was collected and centrifuged at 5,000×g, 4° C., 10 min. The pellet was fixed and prepared for transmission electron microscopy. Images of the mitochondria present in the sample showed that the mitochondria had a round shape with many intact cristae (FIG. 16B). Compare to FIG. 16A which shows mitochondria prepared by Dounce homogenizer method.

### Example 14

### Cell Survival and Differential Lysis Under PCT Conditions

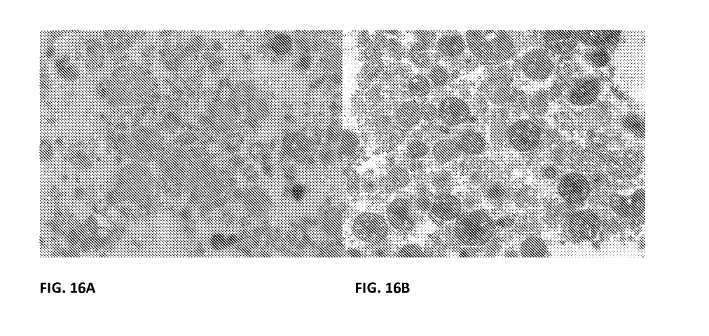

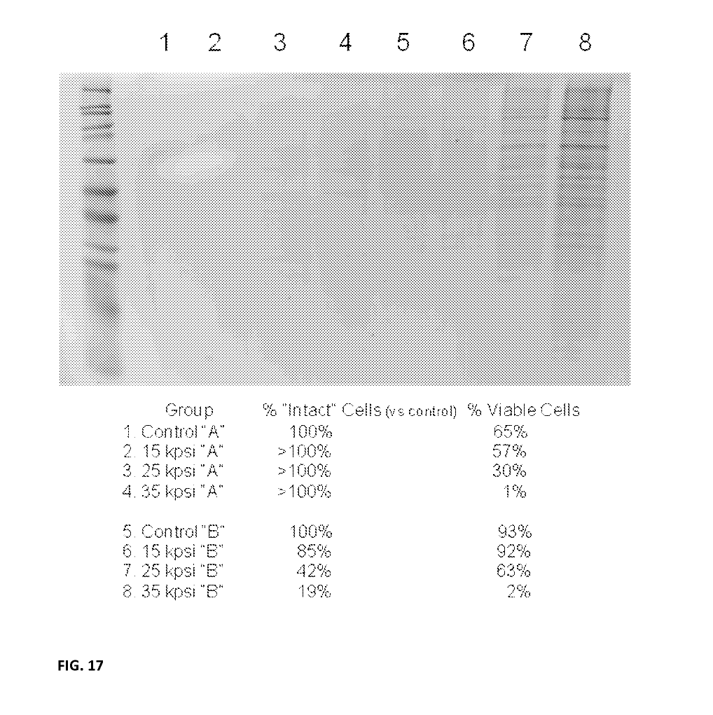

PC12 cells at different growth stages were studied for their responses to PCT-induced lysis. PC12 group “A” cells were from an “aged” culture that had been allowed to overgrow past optimal cell density. PC12 group “B” cells were from a “fresh” healthy culture of cells. Both sets of cells were spun down, washed once with phosphate buffered saline (PBS), and resuspended in PBS prior to pressure cycling. Aliquots of each cell suspension were transferred to PULSE™ tubes and pressurized at the indicated pressure for ten cycles (each cycle consisted of 60 sec at high pressure, followed by 20 sec at atmospheric pressure). Control cells were also transferred to PULSE™ tubes, but were kept at atmospheric pressure throughout the duration of the experiment. After treatment, aliquots of each cell suspension were stained with trypan blue dye for cell count and viability assessment by dye exclusion. The results are shown in FIG. 17. All cell counts include both viable and non-viable cells and are a measure of total intact cells in the sample. Cell counts are expressed as percent of control in that group. Viability is expressed as percent of total cells in each individual group. As expected, the overgrown group “A” control exhibited a reduced proportion of viable cells relative to the healthy Group “B” control.

After removing aliquots for cell counts, the remaining cell suspensions were centrifuged to pellet out the intact cells. The supernatants, containing released cytosolic proteins from the lysed cells were loaded onto SDS-PAGE. Resulting gels were stained with Coomasie Blue dye for visual assessment of protein release from lysed cells. The gel results agree with the results from cell counting; as the number of intact cells goes down, the number of lysed cells, as measured by protein release into the supernatant, increases.

### Example 15

### Isolation of One Cell Type from a Mixed Cell Population by Pressure Cycling: Isolation of Morphologically Intact Sperm Heads from a Mixed Population of Testicular Cells

Mouse testicular tissue was teased apart on ice in phosphate buffered saline using 20 gauge needles. A suspension of sperm and associated somatic cells was collected and placed into a PULSE™ Tube. The sample was processed at 15 kpsi for 20 cycles (20 sec at high pressure followed by 10 sec at atmospheric pressure per cycle). After processing, the sample and an untreated control were evaluated by light microscopy. The control sample contained many somatic cells as well as a large number of intact sperm. The pressure cycling-treated sample contained very few somatic cells, indicating that these cells were efficiently lysed by pressure cycling under these conditions. However, many intact sperm heads were still present after treatment, indicating that sperm heads are more resistant to pressure cycling-induced lysis than are the somatic cells in the suspension.

### Example 16

### Differential Inactivation of Bacterial Strains

It is possible to enrich for one subset of bacteria by using pressure cycling conditions that selectively deplete bacteria of one type, while having less pronounced or no effect on bacteria of another type.

E. coli of two different strains were compared by pressure cycle treating the cultures under a wide range of pressures (from 60 kpsi to 15 kpsi) and with different numbers of cycles (10-100 cycles). It was found that bacteria of strain “A” remained morphologically intact, even under harsh conditions such as 100 cycles at 60 kpsi (30 seconds at high pressure followed by 10 seconds at atmospheric pressure per cycle). Conversely, strain “B”, which is derived from strain “A”, but express different proteins on the membrane, exhibited a different response to pressure cycling. Strain “B” cells were fragmented and lysed by pressure cycling when treated under the conditions of 100 cycles at 60 kpsi. Therefore, if a mixture of strains “A” and “B” together is pulsed under these conditions, Strain “B” cells are lysed and the debris can be easily washed away by centrifugation or other means, allowing for the purification (e.g., enrichment) of bacteria of strain “A”.

### Example 17

### Pressure Cycling-Mediated Depletion of Blood-Derived Proteins from Tissue for Tissue Proteomic Analyses

Blood-derived proteins comprise a significant fraction of total protein extracted from many animal tissues such as skeletal and cardiac muscle. Subjecting the tissue to pressure cycling in a physiological buffer may allow for efficient “squeezing” of the blood components out of the tissue while causing relatively little damage to the bulk of tissue cells. Subsequently, tissue proteins or other components can be extracted in a solution of choice using pressure cycling (or other methods known in the art).

50 mg pieces of rat or mouse heart muscle were placed into PULSE™ Tubes with 1.4 ml of PBS (supplemented with protease inhibitor cocktail) and subjected to 10 rounds of washing by pressure cycling (each round consisted of 15 pressure cycles, 20 sec at 35 kpsi, followed by 20 sec at atmospheric pressure). After each round of PCT the supernatant (wash), containing released blood proteins, was removed and replaced with fresh buffer. After 10 rounds of PCT-mediated washing, the tissue was lysed and total protein was extracted by pressure cycling in either SDS-extraction buffer, or in organic solvent (hexafluoroisopropanol).

The washes and the tissue extract were then analyzed by SDS-PAGE and Western blotting. Coomassie blue staining showed that the first five washes contain the bulk of blood-derived proteins such as serum albumin and hemoglobin as well as a number of other protein bands, while washes 6-10 contain relatively little protein. These results indicate that after 5 rounds of PCT-mediated washing, little additional albumin or hemoglobin could be washed out of the tissue. Western blotting confirmed that high levels of IgG (a blood-derived protein) were present in the first 5 washes, but very little additional IgG could be detected in washes 6-10. Western blotting also showed that IgG contamination could not be detected in the washed heart muscle lysate, but calsequestrin, a muscle-specific marker, was present. These results confirm that under these conditions, 5 rounds of tissue washing by pressure cycling is sufficient to significantly reduce the amount of blood-derived proteins contaminating the tissue extract. In addition, calsequestrin was not detected in the washes, indicating that these washing conditions do not lead to massive disruption of the tissue and significant loss of tissue-specific proteins into the washes.

### Example 18

### Application of Pressure Cycling Technology (PCT) for the Synchronization of Caenorhabditis elegans Cultures

The nematode Caenorhabditis elegans is one of the most extensively studied multi-cellular organisms in biology. It is so well characterized, that wild type adult hermaphrodites are known to be comprised of precisely 959 somatic cells. The nematode is further characterized as having a tough chitinous cuticle that is particularly resistant to disruption.

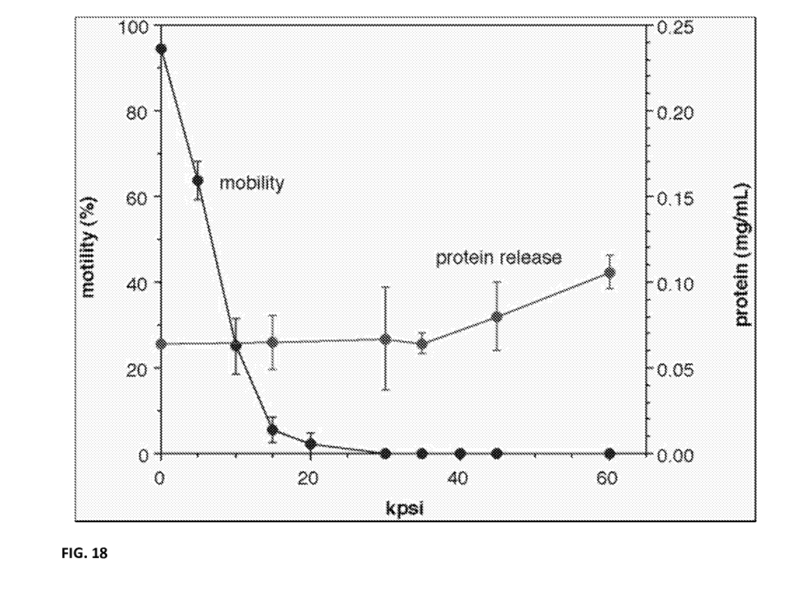

Experiments have primarily aimed at using high pressure to attain complete disruption of nematodes to enable more reliable proteomic and glycoproteomic analysis. It was discovered that nematodes can survive a limited number of pressure cycles up to 20 kpsi with a minimum pressure requirement of 30 kpsi being required for total eradication of nematodes (FIG. 18). By interpolation, a LD₅₀ of 8 kpsi for a minimum of 20 pressure cycles (each cycle for 10 seconds at maximum pressure) was determined.

C. elegans are easily cultured in the laboratory. However, unless synchronized, these cultures exist as a continuum of embryonic, larval stages (L1 through L4), and adult hermaphroditic stage nematodes. Thus, synchronization of cultures becomes necessary to study specific larval stages of the organism. For example, once synchronized, cultures can be arrested at the L1 stage by growth on bacteria-free media. Synchronization necessarily involves the complete eradication of all larval and adult stage nematodes in a manner that only viable embryos are produced. Typically, this is done chemically by incubating the heterogenous population in alkaline hypochlorite solution (140 mM NaOCl, 250 mM KOH) for the destruction and dissolution of nematodes to release viable embryos. The embryos are then recovered centrifugally, but must be copiously washed to remove all traces of hypochlorite before they can be used to produce synchronized cultures.

In our laboratory, we have used “pressure-inactivated” nematode pellets obtained at 30 or 40 kpsi to seed new cultures with apparent synchronization. Microscopic assessment of the pellets, including Trypan Blue permeability assay, has confirmed 100% mortality of both larval and adult nematodes, and regrowth on new plates evidences the viability of residual embryos. However, at 60 kpsi, no regrowth was observed suggesting that at this higher pressure, embryos are effectively destroyed. The proposed method reduces the procedural complexity of chemical methods and eliminates the possibility of carrying over harsh chemicals detrimental to the downstream.

### Example 19

### Isolation of Functional Mitochondria from Fresh Rat Kidney

Highly enriched mitochondrial fractions can be used to examine mitochondrial function in different disease states and to compare mitochondria from different tissues. Functional mitochondria extracted from fresh tissue can be used to provide insights into mitochondrial dysfunction associated with aging, different nutritional states, as well as many diseases.

Mitochondria were isolated from fresh rat kidney tissue as follows: Rats were sacrificed and both kidneys were rapidly removed and placed into ice-cold buffer N1 (250 mM sucrose, 10 mM HEPES, 1 mM EGTA pH 7.4, supplemented with 0.5% fatty acid-free BSA). Minced kidney tissue was split between three PULSE™ Tubes and treated at 10,000 psi for 20 seconds followed by atmospheric pressure for 5 seconds. The pressure cycle was repeated five times at 4° C. After pressure cycling the extracts from the 3 PULSE™ Tubes were pooled and centrifuged at 1000×g for 8 min at 4° C. to pellet cell and tissue debris. The supernatant was centrifuged at 14,000×g for 8 min at 4° C. to pellet the mitochondria-enriched fraction. This mitochondrial pellet was then washed in N1 followed by a wash in N2 buffer (250 mM sucrose, 10 mM HEPES pH 7.4) to remove BSA. The final pellet was suspended in N2 buffer to a final volume of 0.1 ml.

Mitochondrial function was assayed by calculating respiratory control ratios (RCR) and ADP/O ratios. Samples extracted as described above were compared to controls extracted using a standard glass/TEFLON® homogenizer. Mitochondrial respiration was measured using glutamate/malate method. The results shown below in table 4 indicate that mitochondria extracted using pressure cycling exhibit normal respiration parameters and are comparable to control mitochondria extracted using a homogenizer.

TABLE 4
Mitochondrial Function

| | RCR 3/2 | RCR 3/4 | ADP/O |
|---|---|---|---|
| Sample 1 | 6.8 | 4.9 | 2.9 |
| Sample 2 | 6.8 | 4.6 | 2.5 |
| Sample 3 | 6.6 | 4.3 | 2.5 |
| Sample 4 | 6.6 | 3.7 | 2.5 |
| Control 1 | 7.0 | 3.7 | 2.6 |
| Control 2 | 6.7 | 4.6 | 2.7 |

### Example 20

### Isolation of Morphologically Normal Mitochondria from Fresh Rat Kidney Tissue by Pressure Cycling

Mitochondria were isolated from kidney tissue as described in Example 19, above. Control mitochondria were isolated from fresh rat liver using a well-established standard homogenizer protocol. After isolation, 10 μl of the final mitochondrial suspension was fixed for electron microscopy. Transmission electron micrographs are shown at 3000× magnification. Results confirm that mitochondria extracted by pressure cycling exhibit normal morphology (FIGS. 19A and 19B).

### Example 21

### Extraction of RNA from Bone Using Pressure Cycling

RNA can be extracted from small pieces of intact bone without grinding, lysing or dissolving the bone matrix. Small pieces of mouse or bird bone were either freshly harvested or stored in RNALATER™. Pieces of bone (˜1-2 mm³) were placed into 0.15 ml TRIZOL® (Invitrogen) and subjected to pressure at 35,000 psi for 30 cycles (20 seconds at high pressure followed by 5 seconds at ambient pressure per cycle). After pressure cycling the intact bone pieces were removed and discarded. RNA was isolated from the TRIZOL® using the manufacturer's protocol. RNA recovery in most samples was 2-6 μg. RNA purity was confirmed by OD_{260/280} ratio which was in the 1.7-2.0 range for most samples.

### Example 22

### Dose-Response of P19 Embryonal Carcinoma Cells to Increasing Pressure

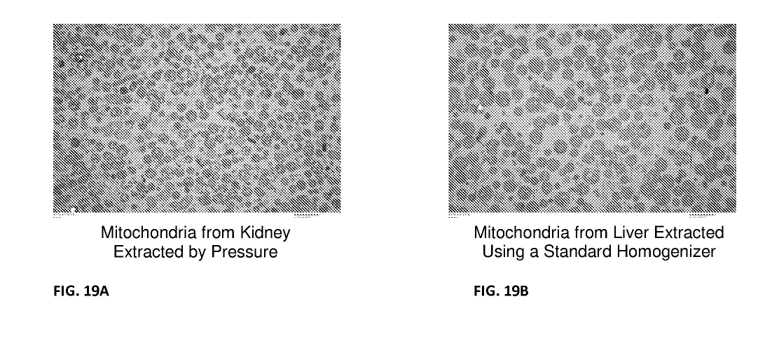

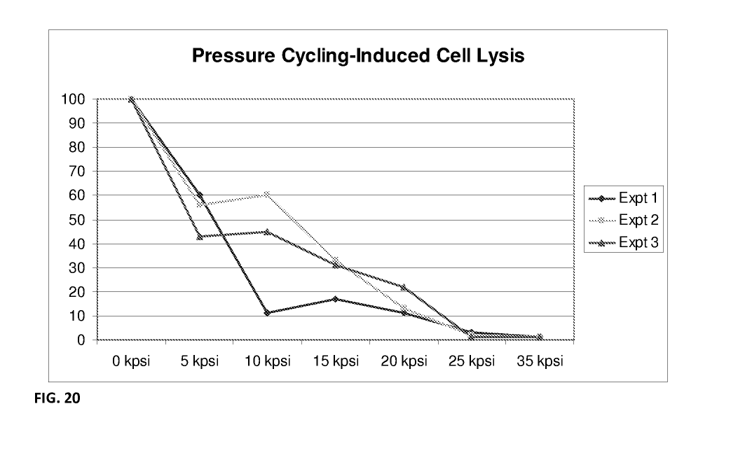

P19 embryonal carcinoma cells were subjected to pressure cycling at different pressures to determine what conditions result in efficient killing of a majority of cells. Cells were subjected to 10 cycles (50 seconds at high pressure followed by 10 seconds at ambient pressure) at 5,000, 10,000, 15,000, 20,000, 25,000 and 35,000 psi. Control cells were treated in an identical manner, but were kept at ambient pressure. After treatment, cells were counted to determine the proportion of cells that had lysed. Results are expressed relative to untreated control (control =100% survival). FIG. 20 shows the results from three independent replicates. The optimum condition with consistent survival of <5% of cells was determined to be 10 cycles at 25,000 psi.

### Example 23

### Cell Culture Recovery after Pressure Cycling

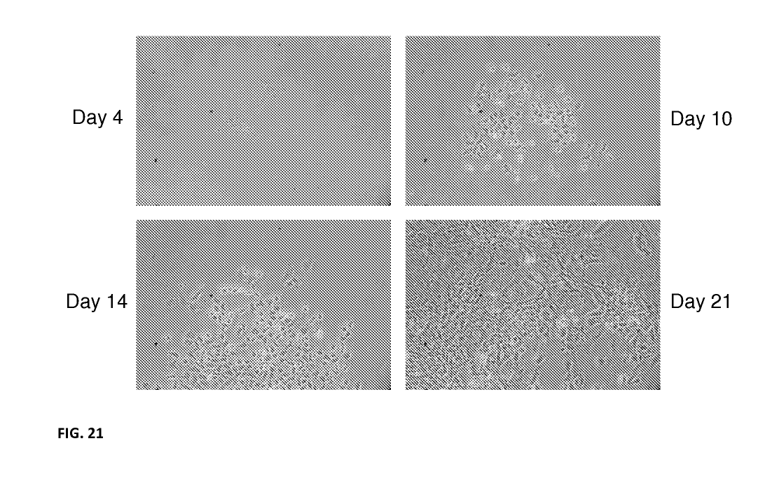

In order to determine whether cells that initially survive pressure cycling at 25,000 psi retain the ability to divide and re-establish cultures, cells were treated as described above at 25,000 psi. After treatment, the surviving cells were seeded into culture and their growth was monitored by light microscopy. Results confirm that after 10 cycles at 25,000 psi, there are a small number of P19 cells that can survive and divide in culture for extended periods of time (FIG. 21).

### Example 24

### Cell Differentiation after Pressure Cycling

P19 embryonal carcinoma cells can differentiate into muscle and neuronal lineages in vitro. Undifferentiated P19 cells were treated by pressure cycling at 24,000 psi to examine the effect of pressurization on spontaneous differentiation. Cells were subjected to 10 cycles of pressure, 50 seconds 24,000 psi followed by 10 seconds at ambient pressure. After treatment, cells were counted to estimate the number of viable cells as determined by Trypan blue exclusion assay. Control unpressurized P19 cells were diluted to match the concentration of viable cells in the treated group (to control for the effect of cell seeding density on subsequent differentiation and morphology). Control and pressurized cells were then seeded into fresh culture media and their growth was monitored by light microscopy.

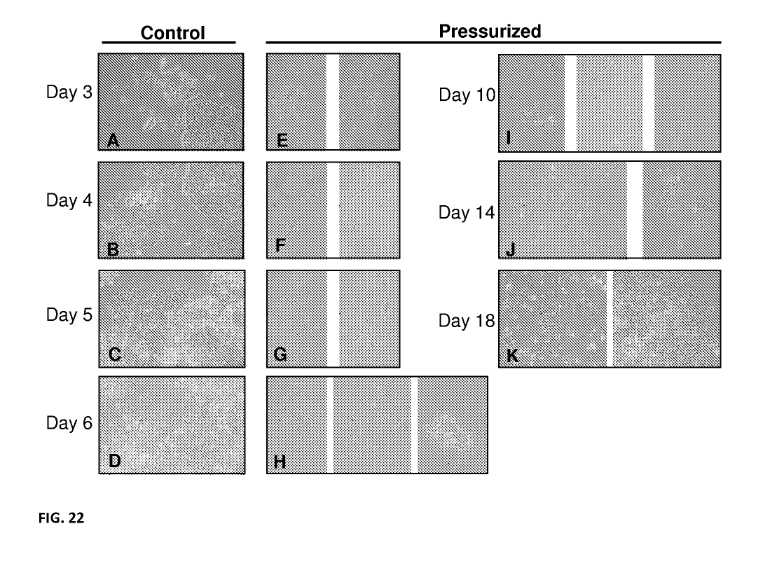

Over the course of the first six days, the control cells exhibit normal undifferentiated morphology and are rapidly dividing (FIG. 22, Panels A, B, C, D). After 6 days the control cells become overgrown and begin to die off.

Pressurized cells are much sparser than the controls, with only individual cells visible in most fields of view (2 or 3 fields of view are shown for all treated timepoints). The morphology of the treated cells is also noticeably different from controls. Treated cells appear “large and flat” (note that the magnification of all images is the same) and begin to exhibit a neuronal-like morphology (see panels F, G, H, I and J). In addition to these neuronal-like cells, patches of undifferentiated cells begin to be apparent by day 6 (panel H, right side). After 2 weeks in culture the cells are quite dense and exhibit areas of both undifferentiated (panel K, right side) and neuronal-like (Panel K, left side) morphology.

### REFERENCES

Lopez M F, Kristal B S, Chemokalskaya E, Lazarev A, Shestopalov A I, Bogdanova A, Robinson M. High-throughput profiling of the mitochondrial proteome using affinity fractionation and automation. Electrophoresis. 2000 (16):3427-40.
Matt P, Fu Z, Fu Q, Van Eyk J E., Biomarker Discovery: Proteome Fractionation and Separation in Biological Samples. Physiol Genomics. 2007 Dec. 27
McDonald T, Sheng S, Stanley B, Chen D, Ko Y, Cole R N, Pedersen P, Van Eyk J E., Expanding the subproteome of the inner mitochondria using protein separation technologies: one- and two-dimensional liquid chromatography and two-dimensional gel electrophoresis. Mol Cell Proteomics. 2006 December; 5(12):2392-411.
Vo T D, Palsson B O., Building the power house: recent advances in mitochondrial studies through proteomics and systems biology. Am J Physiol Cell Physiol. 2007 January; 292(1):C164-77.

A number of embodiments of the invention have been described. Nevertheless, it will be understood that various modifications may be made without departing from the spirit and scope of the invention.

1. A method of extracting a molecular complex from a sample, the method comprising:
providing a mixture at a first pressure, P₀, wherein the mixture comprises a sample and a liquid phase, wherein the sample comprises the molecular complex;
exposing the mixture to a second pressure, P₁, wherein P₁ pressure is greater than P₀;
exposing the mixture to a third pressure, P₂, wherein P₂ is less than P₁; and
fractionating the mixture, thereby extracting the molecular complex from the sample.

2. The method of claim 1, wherein fractionating comprises centrifugation, chromatography, electrophoresis, filtration, or dialysis.

3. The method of claim 1, wherein P₁ is between about 1,000 psi and about 100,000 psi.

4.-6. (canceled)

7. The method of claim 1, wherein P₂ is about equal to P₀.

8. The method of claim 1, wherein P₂ is greater than P₀.

9. The method of claim 1, wherein P₂ is less than P₀.

10.-15. (canceled)

16. The method of claim 1, wherein the sample is exposed to a pressure cycle, wherein P₀, P₁, and P₂ comprise the pressure cycle.

17.-21. (canceled)

22. The method of claim 1, wherein the pressure is applied as hydraulic or pneumatic pressure.

23. The method of claim 1, wherein the method is performed at a temperature between about 0° C. and about +100° C.

24. The method of claim 1, wherein the liquid phase comprises a buffer.

25.-27. (canceled)

28. The method of claim 1, wherein the liquid phase comprises a solvent.

29. The method of claim 1, wherein the liquid phase comprises a protease inhibitor, a DNAse inhibitor, or an RNAse inhibitor.

30. The method of claim 1, wherein the liquid phase comprises a protease, a DNAse, an RNAse, or a lipase.

31. The method of claim 1, wherein the sample is of biological or of synthetic origin.

32. (canceled)

33. The method of claim 1, wherein the sample comprises a cell, a collection of cells, a membrane, a biological sample, or a collection of cells.

34. The method of claim 1, wherein the sample size is from about 10 microliters to about 50 milliliters.

35.-38. (canceled)

39. The method of claim 1, wherein the molecular complex comprises an organelle, a protein complex, a membrane channel, a membrane pore, a transcription factor complex, a signal transduction complex, or a sub-organelle structure.

40.-45. (canceled)

46. The method of claim 1, wherein the extracted molecular complex is further analyzed.

47.-48. (canceled)

49. The method of claim 1, wherein the method further comprises a purification step.

50.-52. (canceled)

53. The method of claim 1, wherein the method comprises an additional fractionation step.

54.-56. (canceled)
